(19) United States
(12) Patent Application Publication
(10) Pub. No.: US 2010/0331678 A1
(43) Pub. Date: Dec. 30, 2010

(54) Multi-Syringe Power Injector using Single Drive Ram

(76) Inventor: Frank M. Fago, Mason, OH (US)

Correspondence Address: Mallinckrodt Inc., 675 McDonnell Boulevard, HAZELWOOD, MO, 63042, US

(21) Appl. No.: 12/919,082

(22) PCT Filed: Feb. 23, 2009
(86) PCT No.: PCT/US09/34827

§ 371 Date: Aug. 24, 2010

(51) Int. Cl. A61B 6/00

(52) U.S. Cl. 600/432

(57) ABSTRACT

A power injector (110) is disclosed that operates a drive train (116) in a single, common configuration to operate each of a first syringe (132) and a second syringe (152). That is, no changes need to be made to the drive train (116) to change the discharge from the first syringe (132) to the second syringe (152), or vice versa. In one embodiment, the first plunger (140) of the first syringe (132) is maintained in a stationary position, while its corresponding first syringe barrel or housing (134) is moved, to provide a discharge from the first syringe (132). Conversely for the noted embodiment, the second plunger (160) of the second syringe (152) is moved, while its corresponding second syringe barrel or housing (154) is maintained in a stationary position.

### RELATED APPLICATIONS

This application claims priority to U.S. provisional application Ser. No. 61/031,026 filed on 25 Feb. 2008 and entitled “MULTI-SYRINGE POWER INJECTOR USING SINGLE DRIVE RAM”.

### FIELD OF THE INVENTION

The present invention generally relates to power injectors and, more particularly to the drive train for operating multiple syringes mounted on a power injector.

### BACKGROUND

Various medical procedures require that one or more medical fluids be injected into the patient. Medical imaging procedures oftentimes involve the injection of a contrast media into the patient, possibly along with saline or other fluids. Other medical procedures involve injecting one or more fluids into a patient for therapeutic purposes. Power injectors may be used for these types of applications.

A power injector generally includes what is commonly referred to as a powerhead. One or more syringes may be mounted to the powerhead in various manners (e.g., detachably; rear-loading; front-loading; side-loading). Each syringe typically includes what may be characterized as a syringe plunger, piston, or the like. Each such syringe plunger is designed to interface with (e.g., contact and/or temporarily interconnect with) an appropriate syringe driver that is incorporated into the powerhead, such that operation of the syringe driver axially advances the associated syringe plunger inside and relative to a barrel of the syringe. One typical syringe driver is in the form of a ram that is mounted on a threaded lead or drive screw. Rotation of the drive screw in one rotational direction advances the associated ram in one axial direction, while rotation of the drive screw in the opposite rotational direction advances the associated ram in the opposite axial direction.

Various types of drive trains have at least been proposed for dual-head power injectors—power injectors where two syringes may be mounted to its powerhead. There are dual-head power injectors that have a separate motor for each syringe. Having completely separate drive trains for each syringe raises the overall cost of the power injector. Other dual-head power injectors use a single motor to drive two different syringes, but at different times. In this case, there may be a “transmission” of sorts to change the drive output from one syringe to another syringe. Moreover, the drive train in this case may have duplicate drive train sections for each of the syringes (e.g., a separate drive screw and ram for each syringe).

### SUMMARY

The first through the eighth aspects of the present invention are each embodied by a power injector. This power injector includes a drive train that is operable in a first configuration. Other components of this power Injector include a first housing, a first plunger that is disposed within and movable relative to this first housing, a second housing, and a second plunger that is disposed within and movable relative to this second housing.

In the case of the first aspect of the present invention, the first housing of the above-noted power injector is a movable structure. One part of an operation of the drive train in its first configuration collectively moves the first housing, the second housing, and the second plunger relative to the first plunger, where the first plunger is maintained in a stationary condition. Another part of the operation of the drive train in the same first configuration moves the second plunger relative to the second housing.

In the case of the second aspect of the present invention, one part of an operation of the drive train in its first configuration moves the first plunger relative to its corresponding first housing, while another part of the operation of the drive train in this same first configuration moves the second plunger relative to its corresponding second housing. Another component of this particular power injector is a discharge sequence controller. This discharge sequence controller allows a discharge from the power injector to be changed from the first housing to the second housing, as well as from the second housing to the first housing.

In the case of the third aspect of the present invention, one part of an operation of the drive train in its first configuration moves the first plunger relative to its corresponding first housing, while another part of the operation of the drive train in this same first configuration moves the second plunger relative to its corresponding second housing. Another component of this particular power injector is a brake. A state of this brake may be changed, to in turn to change a discharge sequence for the power injector between the first and second housings (e.g., to change the discharge sequence from the first housing to the second housing, and vice versa). A non-mechanical signal is used to change the state of the brake,

In the case of the fourth aspect of the present invention, one part of an operation of the drive train in its first configuration moves the first plunger relative to its corresponding first housing, while another part of the operation of the drive train in this same first configuration moves the second plunger relative to its corresponding second housing. Another component of this particular power injector is a brake that is at least operatively interconnected with the first housing. This brake may be changed between disengaged and engaged configurations (from the disengaged configuration to the engaged configuration, and vice versa). The engaged configuration for the brake maintains the first housing in a fixed position.

In the case of the fifth aspect of the present invention, one part of an operation of the drive train in its first configuration moves the first plunger relative to its corresponding first housing, while another part of the operation of the drive train in this same first configuration moves the second plunger relative to its corresponding second housing. Another component of this particular power injector is a brake that is disposable in first and second brake configurations. The first brake configuration allows the first housing and first plunger to move collectively during operation of the drive train in its first configuration. The second brake configuration allows the first plunger to move relative to the first housing during operation of the drive train in its first configuration.

In the case of the sixth aspect of the present invention, one part of an operation of the drive train in its first configuration moves the first plunger relative to its corresponding first housing, while another part of the operation of the drive train in this same first configuration moves the second plunger relative to its corresponding second housing. Another component of this particular power injector is a valve. A first force biases this valve to a position where the valve blocks a flow out of the second housing. This first force is separate from and independent of a pressure in the first housing.

In the case of the seventh aspect of the present invention, one part of an operation of the drive train in its first configuration moves the first plunger relative to its corresponding first housing, while another part of the operation of the drive train in this same first configuration moves the second plunger relative to its corresponding second housing. A first frictional interaction exists between the first housing and its corresponding first plunger. A second frictional interaction exists between the second housing and its corresponding second plunger. The magnitudes of these first and second frictional interactions are different in the case of the seventh aspect.

Various refinements exist of the features noted in relation to each of the above-noted first through the seventh aspects of the present invention. Further features may also be incorporated in each of the above-noted first through the seventh aspects of the present invention as well. These refinements and additional features may exist individually or in any combination in relation to each of the first through the seventh aspects. That is, each of the following features that will be discussed is not required to be used with any other feature or combination of features unless otherwise specified.

A first relative movement between the first plunger and its corresponding first housing may provide a first fluid discharge from the first housing, and a second relative movement between the second plunger and its corresponding second housing may provide a second fluid discharge from the second housing. In one embodiment, these first and second relative movements do not overlap in time—as such the first and second fluid discharges occur at completely independent times. Stated another way, the first and second relative movements may be executed sequentially (immediately following each other or with a time delay therebetween), such that the first and second fluid discharges may occur sequentially. Although the first relative movement could occur before the second relative movement (and thereby the first fluid discharge before the second fluid discharge), the reverse could be true as well. In one embodiment, one part of the operation of the drive train in its first configuration collectively moves the first housing, the second housing, and the second plunger relative to a stationary first plunger, while another part of the operation of the drive train in its first configuration moves the second plunger relative to a stationary second housing, a stationary first housing, and a stationary first plunger.

A valve of any appropriate size, shape, or configuration, and/or type may be utilized by the power injector, for instance a valve that establishes or determines a default order in which fluid is discharged from the first and second housings during operation of the drive train in its first configuration and including for the case where the first and second housings are the same size (e.g., have the same fluid volume). Such a valve may establish a default discharge sequence between the first and second housings. In any case, a number of additional characterizations may be made in relation to this valve. A first force may bias the valve to a position where it blocks a flow out of the second housing, where this first force may be separate from and independent of a pressure in the first housing. That is, the pressure within the first housing need not be greater than the pressure within the second housing to bias the valve to its closed position in this characterization. Any appropriate way of generating a desired magnitude for the first force may be utilized (to bias the valve to a closed position in relation to the second housing), for instance using one or more biasing springs or the biasing members. The biasing force may be of any appropriate magnitude, which of course will affect the time at which the valve opens such that fluid may be discharged from the second housing. Changing the magnitude of the biasing force exerted on the valve may require a greater amount of relative movement between the second plunger and the second housing to open the valve.

The above-noted valve may be characterized as being movable between first and second positions (e.g., to provide discharge and no-discharge conditions or states in relation to the second housing). In one embodiment, a fluid discharge from the second housing is allowed only when the fluid pressure within the second housing generates a force that is greater than the first force (which biases the valve to its closed position in relation to the to second housing) by a certain amount. It should be appreciated that forces in addition to the above-noted first force may be exerted on the valve, and which may affect the fluid pressure within the second housing that will be required to open the valve.

Frictional differences may be utilized to determine a default order in which fluids are discharged from the first and second housings, including for the case where the first and second housings are the same size (e.g., have the same fluid volume). These frictional differences may establish a default discharge sequence between the first and second housings. A first frictional interaction may exist between the first plunger and the first housing, while a second frictional interaction may exist between the second plunger and the second housing, where the magnitude of the first frictional interaction is different from a magnitude of the second frictional interaction. In one embodiment, the magnitude of the first frictional interaction is less than the magnitude of the second frictional action, and this frictional difference may dictate that fluid be discharged from the first housing prior to fluid being discharged from the second housing during operation of the drive train in its first configuration, at least as a default condition. The frictional interaction may oppose relative between a plunger and its corresponding housing.

A first frictional force may exist between the first plunger and the first housing, while a second frictional force may exist between the second plunger and the second housing, where the magnitudes of these first and second frictional forces are different. This frictional force may oppose relative motion between a plunger and its corresponding housing. These different frictional forces may be utilized to determine a default order in which fluids are discharged from the first and second housings, respectively, during operation of the drive train in its first configuration. Different frictional interactions or frictional forces between the first plunger/first housing and second plunger/second housing may be realized in any appropriate manner.

Consider the case where the first and second housings have the same inner diameter, and again where fluid discharges from the first and second housings are provided by a common operation of the drive train in its first configuration. Having different frictional interactions/forces between the first plunger/first housing and second plunger/second housing may be used to establish a default order in which fluid is discharged from the first and second housings. In the case where the first plunger/first housing interface has a smaller frictional interaction/frictional force compared to the second plunger/second housing interface, the fluid discharge from the first housing may be initiated prior to the initiation of the fluid discharge from the second housing. That is, utilizing different frictional interactions/frictional forces between the first plunger/first housing interface and the second plunger/second housing interface may alleviate the need to utilize a valve to establish a default order in which fluid is discharged from the first and second housings.

Another way to establish a default discharge sequence from the first and second housings is based upon the inner diameters or sizes of the first and second housings. Having the first housing be of a smaller inner diameter than the second housing may alone provide a default discharge sequence where fluid is discharged from the first housing prior to the initiation of any fluid discharge from the second housing, all by an operation of the drive train in its first configuration. In one embodiment, utilizing a smaller first housing allows at least substantially all of the fluid to be discharged from the first housing prior to any fluid being discharged from the second housing, again all by an operation of the drive train in its first configuration.

The power injector may include a discharge sequence controller for affecting the order in which fluid is discharged from the first and second housings through operation of the drive train in its first configuration. In one embodiment, the discharge sequence controller is in the form of a brake of any appropriate size, shape, configuration, and/or type. In any case, the discharge sequence controller may be used to over-ride any of the above-noted options for providing a default discharge sequence in relation to the first and second housings, including when the discharge sequence controller is in the form of a brake. It should be appreciated that the discharge sequence controller may be operated to change the default discharge sequence even for the case of the initial discharge from the power injector.

In one embodiment, a state of a brake may be changed, to in turn to change a discharge sequence for the power injector between the first and second housings (e.g., to change the discharge sequence from the first housing to the second housing, and vice versa). A non-mechanical signal (e.g., an electrical signal) may be used to change the state of the brake.

In one embodiment, a brake is at least operatively interconnected or interacts with the first housing. This brake may be changed between disengaged and engaged configurations (from the disengaged configuration to the engaged configuration, and vice versa). The engaged configuration for the brake may maintain the first housing in a fixed position.

In one embodiment, a brake is disposable in each of first and second brake configurations. The first brake configuration allows the first housing and first plunger to move collectively during operation of the drive train in its first configuration. The second brake configuration allows the first plunger to move relative to the first housing during operation of the drive train in its first configuration.

Now referring to the case of the above-noted power injector (set forth in the first paragraph of this Summary) in accordance with the eighth aspect of the present invention, a hollow interior of the first housing is of a first inner diameter, a hollow interior of the second housing is of a second diameter, the second inner diameter is larger than the first inner diameter, the first and second housings are maintained in a fixed position relative to each other at all times, the first and second plungers are disposed in opposing relation, and the power injector further includes a back-drive resistor that is at least operatively associated with the second housing. One part of the operation of the drive train in its first configuration moves the first plunger relative to a stationary first housing (i.e., the first plunger moves while the first housing remains stationary), while another part of the operation of the drive train in this same first configuration moves a stationary second plunger relative to the second housing (i.e., the second housing moves while the second plunger remains stationary).

Various refinements exist of the features noted in relation to the eighth aspect of the present invention. Further features may also be incorporated in the eighth aspect of the present invention as well. These refinements and additional features may exist individually or in any combination in relation to the eighth aspect. One characterization of the back-drive resistor is that it keeps the second plunger from “back-driving” relative to its associated second housing. This “back-drive” may be in the form of a relative movement between the second plunger and the second housing in a direction that retracts (relatively) the second plunger further within the second housing or increases the spacing between the second plunger and a discharge outlet of the second housing. Another characterization is that the back-drive resistor maintains the second plunger and the second housing in a fixed position relative to each other while the first plunger is being moved relative to the stationary first housing. Yet another characterization is that the back-drive resistor provides a resistive force that opposes a movement of the first and second housings in a direction that is directly opposite of the direction that the first plunger is moving during a corresponding portion of the operation of the drive train in its first configuration.

The back-drive resistor of the eighth aspect may be a structure or combination of structures of any appropriate size, shape, configuration, and/or type. The back-drive resistor may be in the form an external stop, abutment, or the like that is engageable with an external flange of the second housing. In one embodiment, a stop, abutment, or the like may be provided on an interior surface of the second housing to limit the range of relative motion between the second plunger and the second housing by being engageable with the second plunger, for instance the “back side” of a piston head of the second plunger (e.g., to establish a fully retracted position of the second plunger within the second housing). A sufficiently large frictional interface or interaction between the second housing and the second plunger may define the back-drive resistor. Another option would be to provide a one-way ratchet between the second plunger and the second housing. In any case, the “position” of the back-drive resistor may be adjustable along the axis of relative motion between the second plunger and the second housing.

Various refinements exist of the features noted in relation to the power injector of each of the above-noted first through the eighth aspects of the present invention. Further features may also be incorporated in each of the above-noted first through the eighth aspects of the present invention as well. These refinements and additional features may exist individually or in any combination in relation to each of the first through the eighth aspects. That is, each of the following features that will be discussed are not required to be used with any other feature or combination of features unless otherwise specified.

The power injector may be of any appropriate size, shape, configuration, and/or type. The power injector may be used for any appropriate application where the delivery of one or more medical fluids is desired, including without limitation any appropriate medical application (e.g., computed tomography or CT imaging; magnetic resonance imaging or MRI; SPECT imaging; PET imaging; X-ray imaging; angiographic imaging; optical imaging; ultrasound imaging). The power injector may be used in conjunction with any component or combination of components, such as an appropriate imaging system (e.g., a CT scanner). For instance, information could be conveyed between any such power injector and one or more other components (e.g., scan delay information, injection start signal, injection rate). Any appropriate shielding may be utilized for a particular application (e.g., shielding that encases or encloses the first and second housings, along with the first and second plungers),

Operation of the same drive train and in the same first configuration is able to provide a first relative movement (between the first syringe plunger and its corresponding first housing), as well as a second relative movement (between the second syringe plunger and its corresponding second housing). That is, separate drive trains are not required to provide the noted first and second relative movements. Moreover, the configuration of a single drive train need not be changed to provide the first relative movement versus the second relative movement, and vice versa.

The drive train may be of any appropriate size, shape, configuration, and/or type. The drive train may utilize one or more drive sources of any appropriate type (e.g., an electric motor, a hydraulic motor, a pneumatic motor, a piezoelectric motor). In one embodiment, the drive train includes a single ram that moves along an axial path. In one embodiment, the drive train includes a single ram that is interconnected with a single threaded drive screw, where rotation of the single drive screw moves the ram along the length dimension of the drive screw (the direction of movement of the ram along the drive screw depending upon the direction of the relative rotational movement between the drive screw and the ram). Movement of this single ram is able to move the first plunger relative to the first housing, and further is able to move the second plunger relative to the second housing.

Any appropriate interaction may be utilized between the drive train and either the first plunger or the second plunger (e.g., mechanical contact; an appropriate coupling (mechanical or otherwise)). The drive train may be capable of providing bi-directional movement (e.g., a movement in one direction for discharging fluid; a movement in a second direction for accommodating a loading of fluid or so as to return to a position for a subsequent fluid discharge operation). In one embodiment, relative movement between the first plunger and the first housing reduces the spacing between the first and second plungers, while a relative movement between the second plunger and the second housing also reduces the spacing between the first and second plungers. Although the drive train may be capable of bi-directional motion (e.g., via axial movement of a ram along a rotating drive screw), it may be such that a relative movement between the first plunger and first housing and a relative movement between the second plunger and the second housing will only provide a corresponding discharge stroke, and not a retraction (a retraction being in the opposite direction of a relative motion that produces a fluid discharge).

Each of the first and second housings may contain any appropriate medical fluid (e.g., contrast media, a radiopharmaceutical, saline, and any combination thereof). In one embodiment, fluid that is discharged from each of the first and second housings is directed into a common conduit that directs the fluid to a desired location (e.g., to a catheter that is inserted into a patient, for instance for injection). One embodiment has the power injector including separate first and second syringes, where the first syringe includes the first housing (e.g., a first syringe barrel), the first plunger, and a first outlet (e.g., a discharge nozzle on an end of the first syringe barrel), where the second syringe includes the second housing (e.g., a second syringe barrel), the second plunger, and a second outlet (e.g., a discharge nozzle on an end of the second syringe barrel), and where a separate connector is fluidly interconnected with each of the first and second outlets (e.g., the first and second syringes may be separate structures that are structurally interconnected by and/or disposed in adjoining relation to a connector of any appropriate size, shape, configuration, and/or type). These syringes may be characterized as being detachably interconnected with the power injector in any appropriate manner. Another embodiment may be of a configuration where the first housing includes a first outlet, where the second housing includes a second outlet, where the first and second housings are adjoined by a connector, where the first and second outlets each fluidly communicate directly with the connector, and where the first housing, the connector, and the second housing are integrally formed (e.g., such that there is no joint of any kind between the first housing and the connector; such that there is no joint of any kind between the second housing and the connector).

A number of characterizations may be made in relation to the first and second housings, which apply individually and in any appropriate combination. The first and second housings may be maintained in a fixed position relative to each other at all times. Therefore, a movement of one housing will simultaneously move the other housing. The first and second housings may be disposed in opposing relation to each other (e.g., such that their corresponding discharge outlets at least generally project toward each other; such that fluid is discharged from the first housing at least initially generally in the direction of the second housing, and such that fluid is discharged from the second housing at least initially generally in the direction of the first housing). The first and second housings may be disposed along a common axis. A first central, longitudinal reference axis that defines the length dimension of the first housing may be collinear with a second central, longitudinal reference axis that defines the length dimension of the second housing. Operation of the drive train in its first configuration may collectively move the first and second housings.

Each of the first and second plungers may be of any appropriate size, shape, configuration, and/or type. The first and second plungers may be disposed along a common axis. A first relative movement between the first plunger and its corresponding first housing may be along an axis that is collinear with an axis along which a second relative movement between the second plunger and its corresponding second housing occurs. The first plunger may be maintained in a fixed position at all times, and the second plunger may be moved by operation of the drive train in its first configuration (except for the case of the eighth aspect, where the first plunger does move as noted above).

Ninth through fourteenth aspects of the present invention are embodied by a method of operation for a power injector. This power injector includes a drive train, a first plunger that is disposed within and movable relative to a first housing, and a second plunger that is disposed within and movable relative to a second housing. The drive train may be operated in a first configuration to provide a first fluid discharge from the first housing, as well as to provide a second fluid discharge from the second housing. That is, no change need be made in the drive train to discharge fluids from each of the first and second housings. More specifically, the first fluid discharge from the first housing is realized using a first part of the operation of the first drive train in its first configuration to provide a first relative movement between the first plunger and the first housing, while the second fluid discharge from the second housing is realized using a second part of the operation of the first drive train in its first configuration to provide a second relative movement between the second plunger and the second housing. “First” and “second” are not terms of order in relation to the ninth through the fourteenth aspects.

In the case of the ninth aspect of the present invention, the second housing and second plunger are collectively moved using the first part of the operation of the drive train in its first configuration, again where this first part also provides a first relative movement between the first plunger and the first housing to discharge fluid from the first housing. That is, the second plunger and the second housing are maintained in a fixed position relative to each other while being collectively moved in the case of the ninth aspect.

In the case of the tenth aspect of the present invention, a first fluid discharge from the first housing is provided, thereafter a second fluid discharge from the second housing is provided, and thereafter a third fluid discharge from the first housing is provided, all again by an operation of the drive train in its first configuration. That is, there are sequential fluid discharges from the first housing, then from the second housing, and then again from the first housing, all by an operation of the drive train in its first configuration (there could be a time delay between fluid discharges from different housings). The third fluid discharge from the first housing may be provided through a third relative movement between the first plunger and the first housing.

In the case of the eleventh aspect of the present invention, a movement of the first housing is restrained. The provision of the second fluid discharge from the second housing may be initiated in response to this restraint.

In the case of the twelfth aspect of the present invention, a flow out of the second housing may be precluded using at least a first force. As such, the fluid being discharged by the power injector at this time will be the first fluid discharge from the first housing, again through operation of the drive train in its first configuration. The provision of the second fluid discharge out of the second housing may be initiated only when the second relative movement between the second plunger and the second housing generates a force that exceeds this first force by a certain amount.

In the case of the thirteenth aspect of the present invention, a default order in which the first relative movement (between the first plunger and the first housing) and the second relative movement (between the second plunger and the second housing) are realized depends upon frictional differences. In one embodiment, this frictional difference is the existence of different frictional interactions between the first plunger and first housing compared to the second plunger and second housing. In another embodiment, this frictional difference is the existence of different frictional forces between the first plunger and first housing compared to the second plunger and second housing. A frictional interaction/force between a plunger and its corresponding housing may oppose a relative movement therebetween that would provide a fluid discharge.

Various refinements exist of the features noted in relation to each of the above-noted ninth through the thirteenth aspects of the present invention. Further features may also be incorporated in each of the above-noted ninth through the thirteenth aspects of the present invention as well. These refinements and additional features may exist individually or in any combination in relation to each of the ninth through the thirteenth aspects. That is, each of the following features that will be discussed is not required to be used with any other feature or combination of features unless otherwise specified.

Although the second part of the operation of the drive train in its first configuration (associated with the second fluid discharge from the second housing) may be preceded in time by the first part of the operation of the first drive train in this same first configuration (associated with the first fluid discharge from the first housing), the reverse could be utilized as well. The second housing and second plunger may be collectively moved using the first part of the operation of the drive train in its first configuration, again where this first part also provides a first relative movement between the first plunger and the first housing to discharge fluid from the first housing. That is, the second plunger and the second housing may be maintained in a fixed position relative to each other while being collectively moved. In one embodiment, the first relative movement between the first plunger and first housing occurs simultaneously with the collective movement of the second plunger and second housing, such as to by maintaining the first and second housings in a fixed position relative to each other at all times. In one embodiment, the first fluid discharge provided by the first relative movement between the first plunger and first housing occurs prior to the second fluid discharge provided by the second relative movement between the second plunger and the second housing. The relative movement between the first plunger and the first housing may be realized by maintaining the first plunger in a fixed or stationary position, including at all times.

The second fluid discharge provided by the second relative movement between the second plunger and the second housing, may occur after the first fluid discharge provided by the first relative movement between the first plunger and the first housing, while a third fluid discharge provided by a third relative movement between the first plunger and the first housing may occur after the second fluid discharge provided by the second relative movement between the second plunger and the second housing. That is, there may be sequential fluid discharges from the first housing, then from the second housing, and then again from the first housing, all by an operation of the drive train in its first configuration (although there could be a time delay between fluid discharges from different housings). The first relative movement may be provided through moving the first housing relative to a stationary first plunger. Further in this regard, a termination of this first relative movement may be realized by stopping the movement of the first housing. In one embodiment, this termination is provided by activating a brake, for instance to exert a braking force (of any appropriate type and in any appropriate manner) on the first housing. In any case, the second fluid discharge may be initiated in response to a termination of the motion of the first housing. Similarly, the third fluid discharge from the first housing may be initiated in response to a termination of the second relative motion between the second plunger and the second housing, for instance to again allow the first housing to move relative to a stationary first plunger. In one embodiment, the termination of the second relative movement between the second plunger and the second housing is realized by disengaging a brake in relation to the first housing.

A movement of the first housing may be restrained at one or more times during the operation of the drive train in its first configuration. The provision of the second fluid discharge from the second housing (via the second relative movement between the second plunger and the second housing) may be initiated in response to this restraint. The provision of the first fluid discharge from the first housing (via the first relative movement between the first plunger and the first housing) may be provided by moving the first housing relative to a stationary first plunger. In one embodiment, restraining the motion of the first housing may be provided by activating a brake of any appropriate size, shape, configuration, and/or type. Consider the case where the initial discharge from the power injector is the first fluid discharge. Restraining the motion of the first housing may be used to terminate the first fluid discharge and initiate the second fluid discharge (from the second housing). Releasing this restraint may again allow the first housing to move relative to a stationary first plunger. The first housing may move simultaneously with a collective movement of the second plunger and the second housing. The movement of the first housing may be restrained/released at various times to change the power injector discharge back and forth between the first and second housings as desired/required. Movement of the first housing may also be restrained such that the initial discharge from the power injector is the second fluid discharge.

A flow out of the second housing may be precluded based upon the existence of a first force during the operation of the drive train in its first configuration. As such, the fluid being discharged by the power injector at this time will be the first fluid discharge from the first housing, again through operation of the drive train in its first configuration. The provision of the second fluid discharge out of the second housing may be initiated only when the second relative movement between the second plunger and the second housing generates a force that exceeds this first force by at least a certain amount. Any appropriate source may be utilized for this first force, including without limitation any appropriate biasing member or combination of biasing members of any appropriate size, shape, configuration, and/or type (e.g., a spring, an elastomeric structure). In one embodiment, the first forces biases a valve into a position that blocks a flow out of the second housing. The flow-blocking position of this valve may be used to dictate a default order in which fluids are discharged from the first and second housings.

A default order in which fluids are discharged from the first and second housings (e.g., a default discharge sequence) may be based upon first and second frictional interfaces between the first plunger/first housing and the second plunger/second housing, respectively. Having the first frictional interface be less than the second frictional interface may allow the first fluid discharge to occur before the second fluid discharge as a default condition.

It should be appreciated that whether a default discharge order or sequence is established by including a biased valve for the second housing or by using different frictional interfaces between the first plunger/first housing and the second plunger/second housing, the above-described “braking” function may be used to override this default discharge order or sequence. In all of these cases, the first and second housings may be of a common size, a common fluid volume, or both (e.g., the outer diameters of the first and second plunger, at the fluid interface, may be the same). Moreover, it should be appreciated that the noted “braking” function may be provided at a time such that the initial discharge from the power injector is other than in accordance with any established default sequence or order.

Now referring to the method in accordance with the above-noted fourteenth aspect of the present invention (recall that the ninth through the fourteenth aspects share a common set of features, and these features are addressed above), the first plunger is moved in a first direction relative to a stationary first housing to provide the first fluid discharge. The first and second housings are collectively biased in a second direction throughout and in response to the provision of the first fluid discharge, where the first and second directions are opposite of each other. However, this bias is opposed throughout the provision of the first fluid discharge such that the first and second housings each remain in a fixed position throughout the provision of the first fluid discharge. The second fluid discharge is provided after the first fluid discharge in the case of the fourteenth aspect, and is provided by the first plunger, first housing, and second housing being collectively moved in the first direction (the same direction that the first plunger moves relative to the first housing in a stationary state to provide the initial first fluid discharge).

Various refinements exist of the features noted in relation to the fourteenth aspect of the present invention. Further features may also be incorporated in the fourteenth aspect of the present invention as well. These refinements and additional features may exist individually or in any combination in relation to the fourteenth aspect. Providing the first fluid discharge may generate a common pressure in each of the first and second housings. As the outer diameter of the second plunger may be larger than the outer diameter of the first plunger, this may generate the above-noted biasing force (the force that biases the first and second housings in the second direction during the first fluid discharge). For instance, having the second plunger being larger than the first plunger and exposing the first and second plungers to a common pressure creates a differential force between the first and second plungers that may bias the first and second housings in the second direction during the first fluid discharge. This biasing force exerted on the first and second housings during the first fluid discharge may be opposed in any appropriate manner, including without limitation in the manner discussed above with regard to the back-drive resistor.

Various refinements exist of the features noted in relation to each of the above-noted ninth through the fourteenth aspects of the present invention. Further features may also be incorporated in each of the above-noted ninth through the fourteenth aspects of the present invention as well. These refinements and additional features may exist individually or in any combination in relation to each of the ninth through the fourteenth aspects. That is, each of the following features that will be discussed is not required to be used with any other feature or combination of features unless otherwise specified.

The operation of the drive train in its first configuration may entail moving a single ram along an axial path and in a first direction. The operation of the drive train in its first configuration may entail rotating a single threaded drive screw, and advancing a single ram along this drive screw in a first direction in response to the rotation. In each case, the ram could interact with the associated plunger in any appropriate manner such that the motion of the ram is translated to the associated plunger. There could be mechanical contact between the ram and the associated plunger, including without limitation a coupled relationship between the ram and the associated plunger. Although the ram may be capable of bi-directional motion, the motion of the ram in one direction many not provide any relative movement between the first plunger and the first housing or between the second plunger and the second housing.

The first and second fluid discharges may occur in non-overlapping relation, including without limitation being sequential (although there could be a time delay between adjacent-in-time fluid discharges from different housings). Except for the case of the fourteenth aspect, these first and second fluid discharges may be provided in any order (i.e., where the first fluid discharge is provided prior to the second fluid discharge, or vice versa). In one embodiment, the first and second fluid discharges are directed into a common conduit that is fluidly interconnected with each of the first and second housings. Fluid flowing through the conduit may be directed to any appropriate location, for instance to a patient for injection.

The relative movements between the first plunger/first housing and second plunger/second housing to provide the first and second fluid discharges, respectively, may be such that the spacing between the first and second plungers is reduced by the first relative movement between the first plunger and first housing, and further such that the second relative movement between the second plunger and the second housing also reduces the spacing between the first and second plungers (e.g., the first and second plungers may be disposed in opposing relation). In one embodiment, the first and second relative movements between the respective first plunger/first housing and second plunger/second housing occur along a common axial path.

Each of the first and second housings may be in the form of a syringe barrel, including where the first plunger and first housing collectively define a first syringe and where the second plunger and second housing collectively define a separate, second syringe, as well as where the first and second housings are part of a common structure (e.g., integrally formed). In one embodiment and except for the case of the fourteenth aspect, the first and second housings are the common size (e.g., a common inner diameter). In another embodiment, the first and second housings are of different sizes (e.g. different inner diameters). In yet another embodiment, the first housing is smaller than the second housing (e.g. having a smaller inner diameter).

The ninth through the fourteenth aspects may be used for any appropriate application, and may be used to discharge any appropriate fluid or combination of different fluids and in any order. At least certain applications may require appropriate shielding. Any appropriate fluid (e.g., contrast media, saline, a radio-pharmaceutical) may be contained within each of the first and second housings. The same or different fluids may be contained in the first and second housings.

### BRIEF DESCRIPTION OF THE FIGURES

FIG. 1 is a schematic of one embodiment of a power injector.

FIG. 2A is a perspective view of one embodiment of a portable stand-mounted, dual-head power injector.

FIG. 2B is an enlarged, partially exploded, perspective view of a powerhead used by the power injector of FIG. 2A.

FIG. 2C is a schematic of one embodiment of a syringe plunger drive assembly used by the power injector of FIG. 2A.

FIG. 3A is a schematic (cutaway side view) of one embodiment of a power injector having a syringe assembly defined by a pair of opposing syringes, where a valve is used to establish a default discharge sequence.

FIG. 3B is an enlarged view of an interconnection of the opposing syringes from the power injector of FIG. 3A.

FIG. 4A is a schematic (cutaway side view) of one embodiment of a power injector having a syringe assembly defined by a pair of opposing syringes, where different frictional interfaces for the two syringes is used to establish a default discharge sequence.

FIG. 4B is a side view of an alternative configuration for the syringe assembly used by the power injector of FIG. 4A.

FIG. 4C is a perspective view of the syringe assembly of FIG. 4B.

FIG. 5 is a schematic (cutaway side view) of one embodiment of a power injector having a syringe assembly defined by a pair of opposing syringes, where a differential sizing between the two syringes is used to establish a default discharge sequence.

FIG. 6 is a schematic (cutaway side view) of one embodiment of a power injector having a syringe assembly defined by a pair of opposing syringes, where the power injector configuration provides a back-drive resistance.

### DETAILED DESCRIPTION

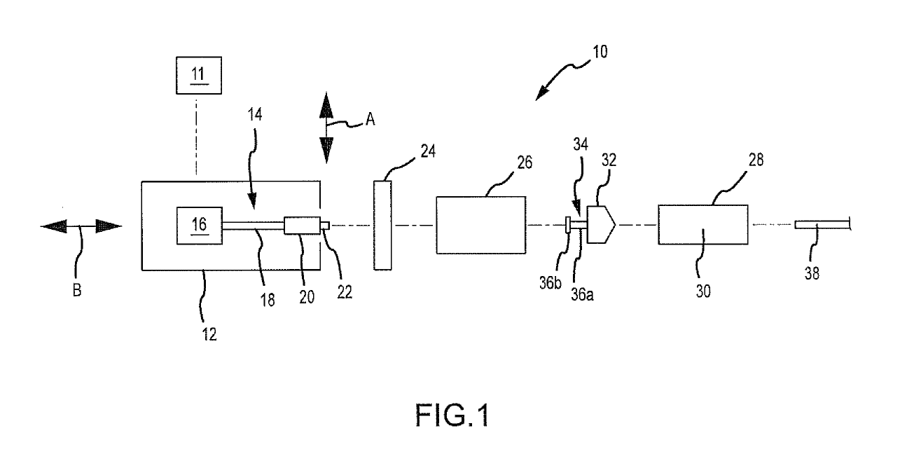

FIG. 1 presents a schematic of one embodiment of a power injector 10 having a powerhead 12. One or more graphical user interfaces or GUIs 11 may be associated with the powerhead 12. Each GUI 11: 1) may be of any appropriate size, shape, configuration, and/or type; 2) may be operatively interconnected with the powerhead 12 in any appropriate manner; 3) may be disposed at any appropriate location; 4) may be configured to provide one or any combination of the following functions: controlling one or more aspects of the operation of the power injector 10; inputting/editing one or more parameters associated with the operation of the power injector 10; and displaying appropriate information (e.g., associated with the operation of the power injector 10); or 5) any combination of the foregoing. Any appropriate number of GUIs 11 may be utilized. In one embodiment, the power injector 10 includes a GUI 11 that is incorporated by a console that is separate from but which communicates with the powerhead 12. In another embodiment, the power injector 10 includes a GUI 11 that is part of the powerhead 12. In yet another embodiment, the power injector 10 utilizes one GUI 11 on a separate console that communicates with the powerhead 12, and also utilizes another GUI 11 that is on the powerhead 12. Each GUI 11 could provide the same functionality or set of functionalities, or the GUIs 11 may differ in at least some respect in relation to their respective functionalities.

A syringe 28 may be installed an this powerhead 12 and, when installed, may be considered to be part of the power injector 10. Some injection procedures may result in a relatively high pressure being generated within the syringe 28. In this regard, it may be desirable to dispose the syringe 28 within a pressure jacket 26. The pressure jacket 26 is typically associated with the powerhead 12 in a manner that allows the syringe 28 to be disposed therein as a part of or after installing the syringe 28 on the powerhead 12. The same pressure jacket 26 will typically remain associated with the powerhead 12, as various syringes 28 are positioned within and removed from the pressure jacket 26 for multiple injection procedures. The power injector 10 may eliminate the pressure jacket 26 if the power injector 10 is configured/utilized for low-pressure injections and/or if the syringe(s) 28 to be utilized with the power injector 10 is (are) of sufficient durability to withstand high-pressure injections without the additional support provided by a pressure jacket 26. In any case, fluid discharged from the syringe 28 may be directed into a conduit 38 of any appropriate size, shape, configuration, and/or type, which may be fluidly interconnected with the syringe 28 in any appropriate manner, and which may direct fluid to any appropriate location (e.g., to a patient).

The powerhead 12 includes a syringe plunger drive assembly or syringe plunger driver 14 that interacts (e.g., interfaces) with the syringe 28 (e.g., a plunger 32 thereof) to discharge fluid from the syringe 28. This syringe plunger drive assembly 14 includes a drive source 16 (e.g., a motor of any appropriate size, shape, configuration, and/or type, optional gearing, and the like) that powers a drive output 18 (e.g., a rotatable drive screw). A ram 20 may be advanced along an appropriate path (e.g., axial) by the drive output 18. The ram 20 may include a coupler 22 for interacting or interfacing with a corresponding portion of the syringe 28 in a manner that will be discussed below.

The syringe 28 includes a plunger or piston 32 that is movably disposed within a syringe barrel 30 (e.g., for axial reciprocation along an axis coinciding with the double-headed arrow B). The plunger 32 may include a coupler 34. This syringe plunger coupler 34 may interact or interface with the ram coupler 22 to allow the syringe plunger drive assembly 14 to retract the syringe plunger 32 within the syringe barrel 30. The syringe plunger coupler 34 may be in the form of a shaft 36a that extends from a body of the syringe plunger 32, together with a head or button 36b. However, the syringe plunger coupler 34 may be of any appropriate size, shape, configuration, and/or type.

Generally, the syringe plunger drive assembly 14 of the power injector 10 may interact with the syringe plunger 32 of the syringe 28 in any appropriate manner (e.g., by mechanical contact; by an appropriate coupling (mechanical or otherwise)) so as to be able to move or advance the syringe plunger 32 (relative to the syringe barrel 30) in at least one direction (e.g., to discharge fluid from the corresponding syringe 28). That is, although the syringe plunger drive assembly 14 may be capable of bi-directional motion (e.g., via operation of the same drive source 16), the power injector 10 may be configured such that the operation of the syringe plunger drive assembly 14 actually only moves each syringe plunger 32 being used by the power injector 10 in only one direction. However, the syringe plunger drive assembly 14 may be configured to interact with each syringe plunger 32 being used by the power injector 10 so as to be able to move each such syringe plunger 32 in each of two different directions (e.g. in different directions along a common axial path).

Retraction of the syringe plunger 32 may be utilized to accommodate a loading of fluid into the syringe barrel 30 for a subsequent injection or discharge, may be utilized to actually draw fluid into the syringe barrel 30 for a subsequent injection or discharge, or for any other appropriate purpose. Certain configurations may not require that the syringe plunger drive assembly 14 be able to retract the syringe plunger 32, in which case the ram coupler 22 and syringe plunger coupler 34 may not be desired. In this case, the syringe plunger drive assembly 14 may be retracted for purposes of executing another fluid delivery operation (e.g., after another pre-filled syringe 28 has been installed). Even when a ram coupler 22 and syringe plunger coupler 34 are utilized, it may such that these components may or may not be coupled when the ram 20 advances the syringe plunger 32 to discharge fluid from the syringe 28 (e.g., the ram 20 may simply “push on” the syringe plunger coupler 34 or on a proximal end of the syringe plunger 32). Any single motion or combination of motions in any appropriate dimension or combination of dimensions may be utilized to dispose the ram coupler 22 and syringe plunger coupler 34 in a coupled state or condition, to dispose the ram coupler 22 and syringe plunger coupler 34 in an un-coupled state or condition, or both.

The syringe 28 may be installed on the powerhead 12 in any appropriate manner. For instance, the syringe 28 could be configured to be installed directly on the powerhead 12. In the illustrated embodiment, a housing 24 is appropriately mounted on the powerhead 12 to provide an interface between the syringe 28 and the powerhead 12. This housing 24 may be in the form of an adapter to which one or more configurations of syringes 28 may be installed, and where at least one configuration for a syringe 28 could be installed directly on the powerhead 12 without using any such adapter. The housing 24 may also be in the form of a faceplate to which one or more configurations of syringes 28 may be installed. In this case, it may be such that a faceplate is required to install a syringe 28 on the powerhead 12—the syringe 28 could not be installed on the powerhead 12 without the faceplate. When a pressure jacket 26 is being used, it may be installed on the powerhead 12 in the various manners discussed herein in relation to the syringe 28, and the syringe 28 will then thereafter be installed in the pressure jacket 26.

The housing 24 may be mounted on and remain in a fixed position relative to the powerhead 12 when installing a syringe 28. Another option is to movably interconnect the housing 24 and the powerhead 12 to accommodate installing a syringe 28. For instance, the housing 24 may move within a plane that contains the double-headed arrow A to provide one or more of coupled state or condition and an un-coupled state or condition between the ram coupler 22 and the syringe plunger coupler 34.

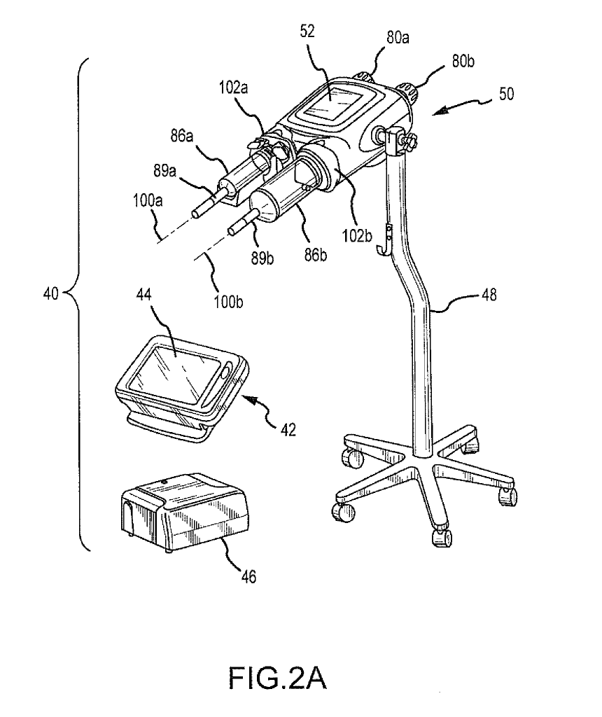

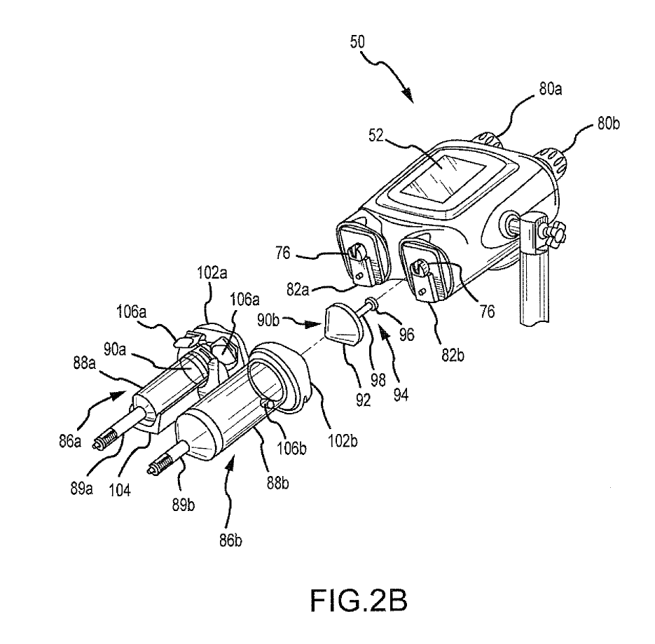

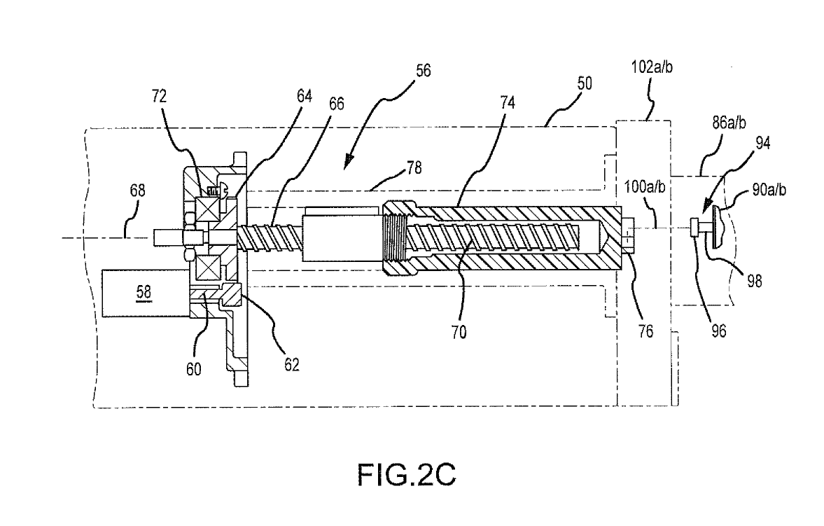

One particular power injector configuration is illustrated in FIG. 2A, is identified by a reference numeral 40, and is at least generally in accordance with the power injector 10 of FIG. 1. The power injector 40 includes a powerhead 50 that is mounted on a portable stand 48. A pair of syringes 86a, 86b for the power injector 40 is mounted on the powerhead 50. Fluid may be discharged from the syringes 86a, 86b during operation of the power injector 40.

The portable stand 48 may be of any appropriate size, shape, configuration, and/or type. Wheels, rollers, casters, or the like may be utilized to make the stand 48 portable. The powerhead 50 could be maintained in a fixed position relative to the portable stand 48. However, it may be desirable to allow the position of the powerhead 50 to be adjustable relative to the portable stand 48 in at least some manner. For instance, it may be desirable to have the powerhead 50 in one position relative to the portable stand 48 when loading fluid into one or more of the syringes 86a, 86b, and to have the powerhead 50 in a different position relative to the portable stand 48 for performance of an injection procedure. In this regard, the powerhead 50 may be movably interconnected with the portable stand 48 in any appropriate manner (e.g., such that the powerhead 50 may be pivoted through at least a certain range of motion, and thereafter maintained in the desired position).

It should be appreciated that the powerhead 50 could be supported in any appropriate manner for providing fluid. For instance, instead of being mounted on a portable structure, the powerhead 50 could be interconnected with a support assembly, that in turn is mounted to an appropriate structure (e.g., ceiling, wall, floor). Any support assembly for the powerhead 50 may be positionally adjustable in at least some respect (e.g., by having one or more support sections that may be repositioned relative to one more other support sections), or may be maintained in a fixed position. Moreover, the powerhead 50 may be integrated with any such support assembly so as to either be maintained in a fixed position or so as to be adjustable relative the support assembly.

The powerhead 50 includes a graphical user interface or GUI 52. This GUI 52 may be configured to provide one or any combination of the following functions: controlling one or more aspects of the operation of the power injector 40; inputting/editing one or more parameters associated with the operation of the power injector 40; and displaying appropriate information (e.g., associated with the operation of the power injector 40). The power injector 40 may also include a console 42 and powerpack 46 that each may be in communication with the powerhead 50 in any appropriate manner (e.g., via one or more cables), that may be placed on a table or mounted on an electronics rack in an examination room or at any other appropriate location, or both. The powerpack 46 may include one or more of the following and in any appropriate combination: a power supply for the injector 40; interface circuitry for providing communication between the console 42 and powerhead 50; circuitry for permitting connection of the power injector 40 to remote units such as remote consoles, remote hand or foot control switches, or other original equipment manufacturer (OEM) remote control connections (e.g., to allow for the operation of power injector 40 to be synchronized with the x-ray exposure of an imaging system); and any other appropriate componentry. The console 42 may include a touch screen display 44, which in turn may provide one or more of the following functions and in any appropriate combination: allowing an operator to remotely control one or more aspects of the operation of the power injector 40; allowing an operator to enter/edit one or more parameters associated with the operation of the power injector 40; allowing an operator to specify and store programs for automated operation of the power injector 40 (which can later be automatically executed by the power injector 40 upon initiation by the operator); and displaying any appropriate information relation to the power injector 40 and including any aspect of its operation.

Various details regarding the integration of the syringes 86a, 86b with the powerhead 50 are presented in FIG. 2B. Each of the syringes 86a, 86b includes the same general components. The syringe 86a includes plunger or piston 90a that is movably disposed within a syringe barrel 88a. Movement of the plunger 90a along an axis 100a (FIG. 2A) via operation of the powerhead 50 will discharge fluid from within a syringe barrel 88a through a nozzle 89a of the syringe 86a. An appropriate conduit (not shown) will typically be fluidly interconnected with the nozzle 89a in any appropriate manner to direct fluid to a desired location (e.g., a patient). Similarly, the syringe 86b includes plunger or piston 90b that is movably disposed within a syringe barrel 88b. Movement of the plunger 90b along an axis 100b (FIG. 2A) via operation of the powerhead 50 will discharge fluid from within the syringe barrel 88b through a nozzle 89b of the syringe 86b. An appropriate conduit (not shown) will typically be fluidly interconnected with the nozzle 89b in any appropriate manner to direct fluid to a desired location (e.g., a patient).

The syringe 86a is interconnected with the powerhead 50 via an intermediate faceplate 102a. This faceplate 102a includes a cradle 104 that supports at least part of the syringe barrel 88a, and which may provide/accommodate any additional functionality or combination of functionalities. A mounting 82a is disposed on and is fixed relative to the powerhead 50 for interfacing with the faceplate 102a. A ram coupler 76 of a ram 74 (FIG. 2C), which are each part of a syringe plunger drive assembly or syringe plunger driver 56 (FIG. 2C) for the syringe 86a, is positioned in proximity to the faceplate 102a when mounted on the powerhead 50. Details regarding the syringe plunger drive assembly 56 will be discussed in more detail below in relation to FIG. 2C. Generally, the ram coupler 76 may be coupled with the syringe plunger 90a of the syringe 86a, and the ram coupler 76 and ram 74 (FIG. 2C) may then be moved relative to the powerhead 50 to move the syringe plunger 90a along the axis 100a (FIG. 2A). It may be such that the ram coupler 76 is engaged with, but not actually coupled to, the syringe plunger 90a when moving the syringe plunger 90a to discharge fluid through the nozzle 89a of the syringe 86a.

The faceplate 102a may be moved at least generally within a plane that is orthogonal to the axes 100a, 100b (associated with movement of the syringe plungers 90a, 90b, respectively, and illustrated in FIG. 2A), both to mount the faceplate 102a on and remove the faceplate 102a from its mounting 82a on the powerhead 50. The faceplate 102a may be used to couple the syringe plunger 90a with its corresponding ram coupler 76 on the powerhead 50. In this regard, the faceplate 102a includes a pair of handles 106a. Generally and with the syringe 86a being initially positioned within the faceplate 102a, the handles 106a may be moved to in turn move/translate the syringe 86a at least generally within a plane that is orthogonal to the axes 100a, 100b (associated with movement of the syringe plungers 90a, 90b, respectively, and illustrated in FIG. 2A). Moving the handles 106a to one position moves/translates the syringe 86a (relative to the faceplate 102a) in an at least generally downward direction to couple its syringe plunger 90a with its corresponding ram coupler 76. Moving the handles 106a to another position moves/translates the syringe 86a (relative to the faceplate 102a) in an at least generally upward direction to uncouple its syringe plunger 90a from its corresponding ram coupler 76.

The syringe 86b is interconnected with the powerhead 50 via an intermediate faceplate 102b. A mounting 82b is disposed on and is fixed relative to the powerhead 50 for interfacing with the faceplate 102b. A ram coupler 76 of a ram 74 (FIG. 2C), which are each part of a syringe plunger drive assembly 56 for the syringe 86b, is positioned in proximity to the faceplate 102b when mounted to the powerhead 50. Details regarding the syringe plunger drive assembly 56 again will be discussed in more detail below in relation to FIG. 2C. Generally, the ram coupler 76 may be coupled with the syringe plunger 90b of the syringe 86b, and the ram coupler 76 and ram 74 (FIG. 2C) may be moved relative to the powerhead 50 to move the syringe plunger 90b along the axis 100b (FIG. 2A). It may be such that the ram coupler 76 is engaged with, but not actually coupled to, the syringe plunger 90b when moving the syringe plunger 90b to discharge fluid through the nozzle 89b of the syringe 86b.

The faceplate 102b may be moved at least generally within a plane that is orthogonal to the axes 100a, 100b (associated with movement of the syringe plungers 90a, 90b, respectively, and illustrated in FIG. 2A), both to mount the faceplate 102b on and remove the faceplate 102b from its mounting 82b on the powerhead 50. The faceplate 102b also may be used to couple the syringe plunger 90b with its corresponding ram coupler 76 on the powerhead 50. In this regard, the faceplate 102b may include a handle 106b. Generally and with the syringe 86b being initially positioned within the faceplate 102b, the syringe 86b may be rotated along its long axis 100b (FIG. 2A) and relative to the faceplate 102b. This rotation may be realized by moving the handle 106b, by grasping and turning the syringe 86b, or both. In any case, this rotation moves/translates both the syringe 86b and the faceplate 102b at least generally within a plane that is orthogonal to the axes 100a, 100b (associated with movement of the syringe plungers 90a, 90b, respectively, and illustrated in FIG. 2A). Rotating the syringe 86b in one direction moves/translates the syringe 86b and faceplate 102b in an at least generally downward direction to couple the syringe plunger 90b with its corresponding ram coupler 76. Rotating the syringe 86b in the opposite direction moves/translates the syringe 86b and faceplate 102b in an at least generally upward direction to uncouple its syringe plunger 90b from its corresponding ram coupler 76.

As illustrated in FIG. 2B, the syringe plunger 90b includes a plunger body 92 and a syringe plunger coupler 94. This syringe plunger coupler 94 includes a shaft 98 that extends from the plunger body 92, along with a head 96 that is spaced from the plunger body 92. Each of the ram couplers 76 includes a larger slot that is positioned behind a smaller slot on the face of the ram coupler 76. The head 96 of the syringe plunger coupler 94 may be positioned within the larger slot of the ram coupler 76, and the shaft 98 of the syringe plunger coupler 94 may extend through the smaller slot on the face of the ram coupler 76 when the syringe plunger 90b and its corresponding ram coupler 76 are in a coupled state or condition. The syringe plunger 90a may include a similar syringe plunger coupler 94 for interfacing with its corresponding ram coupler 76.

The powerhead 50 is utilized to discharge fluid from the syringes 86a, 86b in the case of the power injector 40. That is, the powerhead 50 provides the motive force to discharge fluid from each of the syringes 86a, 86b. One embodiment of what may be characterized as a syringe plunger drive assembly or syringe plunger driver is illustrated in FIG. 2C, is identified by reference numeral 56, and may be utilized by the powerhead 50 to discharge fluid from each of the syringes 86a, 86b. A separate syringe plunger drive assembly 56 may be incorporated into the powerhead 50 for each of the syringes 86a, 86b. In this regard and referring back to FIGS. 2A-B, the powerhead 50 may include hand-operated knobs 80a and 80b for use in separately controlling each of the syringe plunger drive assemblies 56.

Initially and in relation to the syringe plunger drive assembly 56 of FIG. 2C, each of its individual components may be of any appropriate size, shape, configuration and/or type. The syringe plunger drive assembly 56 includes a motor 58, which has an output shaft 60. A drive gear 62 is mounted on and rotates with the output shaft 60 of the motor 58. The drive gear 62 is engaged or is at least engageable with a driven gear 64. This driven gear 64 is mounted on and rotates with a drive screw or shaft 66. The axis about which the drive screw 66 rotates is identified by reference numeral 68. One or more bearings 72 appropriately support the drive screw 66.

A carriage or ram 74 is movably mounted on the drive screw 66. Generally, rotation of the drive screw 66 in one direction axially advances the ram 74 along the drive screw 66 (and thereby along axis 68) in the direction of the corresponding syringe 86a/b, while rotation of the drive screw 66 in the opposite direction axially advances the ram 74 along the drive screw 66 (and thereby along axis 68) away from the corresponding syringe 86a/b. In this regard, the perimeter of at least part of the drive screw 66 includes helical threads 70 that interface with at least part of the ram 74. The ram 74 is also movably mounted within an appropriate bushing 78 that does not allow the ram 74 to rotate during a rotation of the drive screw 66. Therefore, the rotation of the drive screw 66 provides for an axial movement of the ram 74 in a direction determined by the rotational direction of the drive screw 66.

The ram 74 includes a coupler 76 that that may be detachably coupled with a syringe plunger coupler 94 of the syringe plunger 90a/b of the corresponding syringe 86a/b. When the ram coupler 76 and syringe plunger coupler 94 are appropriately coupled, the syringe plunger 90a/b moves along with ram 74. FIG. 2C illustrates a configuration where the syringe 86a/b may be moved along its corresponding axis 100a/b without being coupled to the ram 74. When the syringe 86a/b is moved along its corresponding axis 100a/b such that the head 96 of its syringe plunger 90a/b is aligned with the ram coupler 76, but with the axes 68 still in the offset configuration of FIG. 2C, the syringe 86a/b may be translated within a plane that is orthogonal to the axis 68 along which the ram 74 moves. This establishes a coupled engagement between the ram coupler 76 and the syringe plunger coupler 96 in the above-noted manner.

The power injectors 10, 40 of FIGS. 1 and 2A-C each may be used for any appropriate application, including without limitation for medical imaging applications where fluid is injected into a subject (e.g., a patient). Representative medical imaging applications for the power injectors 10, 40 include without limitation computed tomography or CT imaging, magnetic resonance imaging or MRI, SPECT imaging, PET imaging, X-ray imaging, angiographic imaging, optical imaging, and ultrasound imaging. The power injectors 10, 40 each could be used alone or in combination with one or more other components. The power injectors 10, 40 each may be operatively interconnected with one or more components, for instance so that information may be conveyed between the power injector 10, 40 and one or more other components (e.g., scan delay information, injection start signal, injection rate).

Any number of syringes may be utilized by each of the power injectors 10, 40, including without limitation single-head configurations (for a single syringe) and dual-head configurations (for two syringes). In the case of a multiple syringe configuration, each power injector 10, 40 may discharge fluid from the various syringes in any appropriate manner and according to any timing sequence (e.g., sequential discharges from two or more syringes, simultaneous discharges from two or more syringes, or any combination thereof). Each such syringe utilized by each of the power injectors 10, 40 may include any appropriate fluid, for instance contrast media, a radiopharmaceutical, saline, and any combination thereof. Each such syringe utilized by each of the power injectors 10, 40 may be installed in any appropriate manner (e.g., rear-loading configurations may be utilized; front-loading configurations may be utilized; side-loading configurations may be utilized).

Various embodiments of power injectors will now be described where operation of a single drive train in an unchanged configuration may be used to discharge fluid from multiple syringes. Each of the components of these power injectors may be formed from any appropriate material or combination of materials, unless otherwise noted.

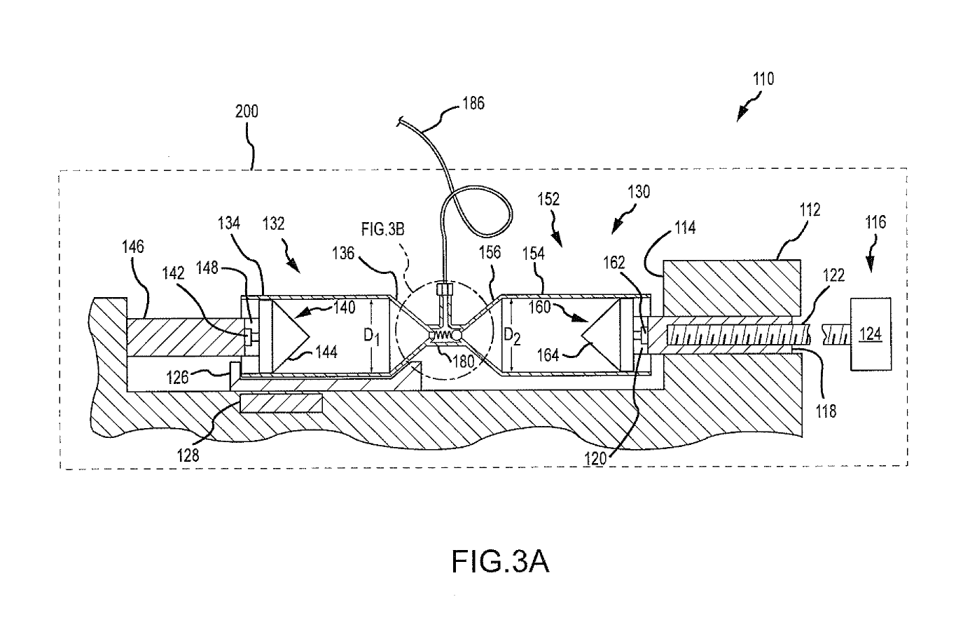

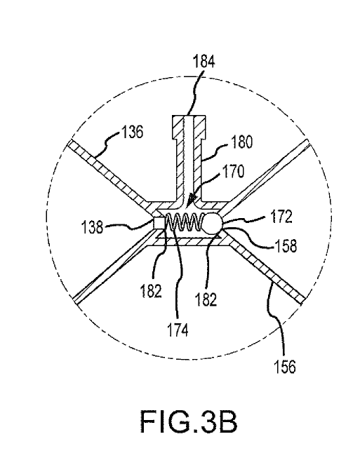

One embodiment of a power injector is illustrated in FIGS. 3A-B, is identified by reference numeral 110, and utilizes a valve 170 to establish a default discharge sequence between a pair of syringes 132, 152. The power injector 110 includes a powerhead 112 of any appropriate size, shape, configuration, and/or type (only schematically illustrated). A receptacle 114 of the powerhead 112 receives what may be characterized as a syringe assembly 130.

The syringe assembly 130 includes a first syringe 132 and a second syringe 152 that are disposed in opposing relation. Components of the first syringe 132 include a first housing or syringe barrel 134, along with a first plunger 140 that is disposed within and movable relative to the first housing 134. A nozzle 136 is included on or extends from an end of the first housing 134. Each of the various components of the first syringe 132 may be of any appropriate size, shape, configuration, and/or type.

A first outlet 138 is included on the end of the nozzle 136 to accommodate fluid discharges from the first syringe 132. Relative movement between the first plunger 140 and the first housing 134 provides a fluid discharge from the first housing 134 through the first outlet 138. In the illustrated embodiment, the first housing 134 is moved relative to a stationary first plunger 140 in a manner that will be discussed in more detail below to discharge fluid from the first housing 134 (e.g., the first plunger 140 may be maintained in a fixed position at all times). Generally, a conical surface of a head 144 of the first plunger 140 interfaces with fluid within the first housing 134 to “push” fluid out of the first housing 134 during the noted relative movement.

The first housing 134 of the first syringe 132 is received within a syringe carriage 126 that is movable relative to the powerhead 112. An optional track, guide, or the like may be used to limit the motion between the syringe carriage 126 and the powerhead 112 to being along an axial path. The first housing 134 could also be movably interconnected directly with the powerhead 112 (e.g., by incorporating an appropriate change in the configuration of the first housing 134). A stationary ram 146 associated with the powerhead 112 may mechanically engage or otherwise interact with the first plunger 140 so that the first plunger 140 may not be moved to the left in the view presented in FIG. 3A. It may be desirable to couple the stationary ram 146 with the first plunger 140 in any appropriate manner. The illustrated embodiment has the first plunger 140 including a coupler 142 that engages a coupler 148 on the end of the stationary ram 146 to provide a mechanical coupling between the stationary ram 146 and the first plunger 140. Providing an appropriate coupling between the stationary ram 146 and the first plunger 140 may be beneficial for one or more purposes, for instance so as to limit or restrain relative motion between the stationary ram 146 and the first plunger 140 in at least one dimension (e.g., so as to not allow the first plunger 140 to move to the right and away from the stationary ram 146 in the view shown in FIG. 3A), to allow the stationary ram 146 to be re-used, and/or the syringe assembly 130 to be in the form of a single-use unit, or the like. Although the stationary ram 146 and the first plunger 140 may be separate structures, they could simply be different parts of single, common structure (e.g., not separable).

Components of the second syringe 152 include a second housing or syringe barrel 154, along with a second plunger 160 that is disposed within and movable relative to the second housing 154. A nozzle 156 is included on or extends from an end of the second housing 154. Each of the various components of the second syringe 152 may be of any appropriate size, shape, configuration, and/or type.

A second outlet 158 is included on the end of the nozzle 156 to accommodate fluid discharges from the second syringe 152. Relative movement between the second plunger 160 and the second housing 154 provides a fluid discharge from the second housing 154 through the second outlet 158. In the illustrated embodiment, the second plunger 160 is moved relative to a then stationary second housing 154 in a manner that will be discussed in more detail below. Generally, a conical surface of a head 164 of the second plunger 160 interfaces with fluid within the second housing 154 to “push” fluid out of the second housing 154 during the noted relative movement. The opposite end of the second plunger 160 may include a coupler 162 for interfacing with the drive train 116 of the power injector 110.

The drive train 116 may include one or more drive sources 124 of any appropriate size, shape, configuration, and/or type (e.g., an electric motor, a hydraulic motor, a pneumatic motor, a piezoelectric motor). The output of at least one drive source 124 rotates a single drive screw 122 (external threads) of the drive train 116. A single ram 118 of the drive train 116 is threadably interconnected with the drive screw 122 in any appropriate manner such that rotation of the drive screw 122 in one rotational direction advances the ram 118 along the drive screw 122 in one direction (e.g., along an axial path), while rotation of the drive screw 122 in the opposite rotational direction advances the ram 118 along the drive screw 122 in the opposite direction (e.g., along an axial path). All that is required is for there to be relative rotational movement between the ram 118 and the drive screw 122 to provide relative axial movement between the ram 118 and the drive screw 122, and this relative rotational movement may be provided in any appropriate manner.

It may be desirable to provide a coupling of any appropriate type between the ram 118 and the second plunger 160, although any appropriate interaction may be utilized to transfer motion from the ram 118 to the second plunger 160 to move the second plunger 160 in at least one direction for discharging fluid from the power injector 110. A mechanical coupling is utilized by the illustrated embodiment, and is schematically illustrated by a coupler 120 of the ram 118 that may couple with the coupler 162 of the second plunger 160. Providing an appropriate coupling between the ram 118 and the second plunger 160 may be used to allow the ram 118 to move the second plunger 160 in opposite directions along a common axial path (i.e., via bidirectional movement of the ram 118). A relative movement between the second plunger 160 and the second housing 154 along an axial path and in one direction may discharge fluid from the second housing 154, while a relative movement between the second plunger 160 and the second housing 154 along an axial path and in the opposite direction may retract the second plunger 160 (e.g., so as to load fluid in or at least accommodate the loading of fluid into the second housing 154; to reposition the ram 118 for a subsequent fluid discharge operation). It should be appreciated that a coupling between the ram 118 and the second plunger 160 may not be required if all that is required by a particular application is for the ram 118 to be able to advance the second plunger 160 for a discharge stroke. That is, although the ram 118 may be capable of bi-directional motion, the ram 118 may in fact only move the second plunger 160 in a single direction.

A number of characterizations may be made in relation to the arrangement of the first syringe 132 and the second syringe 152 in the case of the power injector 110, which apply individually and in any combination. The first syringe 132 and the second syringe 152 may be characterized as being disposed in opposing relation. The first outlet 138 of the first syringe 132 may project toward or face the second outlet 158 of the second syringe 152. The first syringe 132 may be arranged relative to the second syringe 152 such that the spacing between the first plunger 140 and the second plunger 160 decreases whether fluid is being discharged from the first syringe 132 or the second syringe 152 (e.g., the spacing between the plungers 140, 160 may be decreased by a relative movement between the first plunger 140 and the first housing 134 that provides a fluid discharge out of the first housing 134, as well as by a relative movement between the second plunger 160 and the second housing 154 that provides a fluid discharge out of the second housing 154). A central, longitudinal reference axis of the first syringe 132 may be coaxial with a central, longitudinal reference axis of the second syringe 152 (e.g., the syringes 132, 152 may be axially aligned).

The first housing 134 of the first syringe 132 and the second housing 154 of the second syringe 152 may be characterized as being of the same size. In one embodiment, each of the first housing 134 and the second housing 154 has the same fluid volume. In one embodiment, the inner diameter D₁ of the first housing 134 is equal to the inner diameter D₂ of the second housing 154. Stated another way, the outer diameter of the first plunger 140 and the outer diameter of the second plunger 160 may be the same. For purposes of the power injector 110, the diameter D₂ of the second housing 154 (second syringe 152) should be at least as large as the diameter D₁ of the first housing 134 (first syringe 132).

Each of the first syringe 132 and the second syringe 152 discharge into a common connector 180, which may be characterized as being part of the syringe assembly 130, for instance an adjoining structure to each of the first housing 134 and the second housing 154. The connector 180 may be of any appropriate size, shape, configuration, and/or type. The first outlet 138 of the first syringe 132 is fluidly interconnected with one inlet port 182 of the connector 180, while the second outlet 158 of the second syringe 152 is fluidly interconnected with another inlet port 182 of the connector 180. The connector 180 also includes an outlet port 184 in fluid communication with a conduit 186. This conduit 186 may be of any appropriate size, shape, configuration, and/or type (e.g., medical tubing), and may direct fluid to any appropriate fluid target. In one embodiment, the conduit 186 extends to a patient (e.g., a human; an animal) for injection into the patient via a catheter or the like.

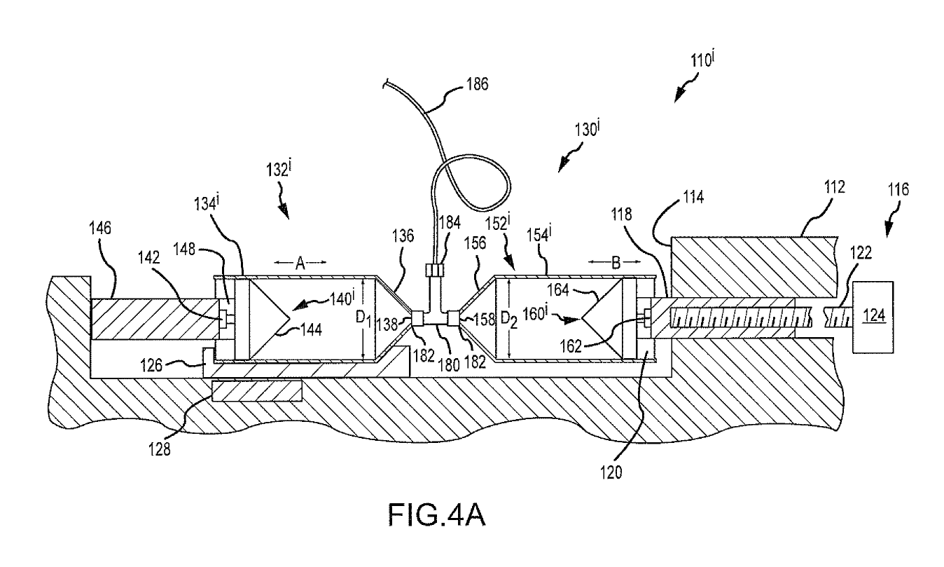

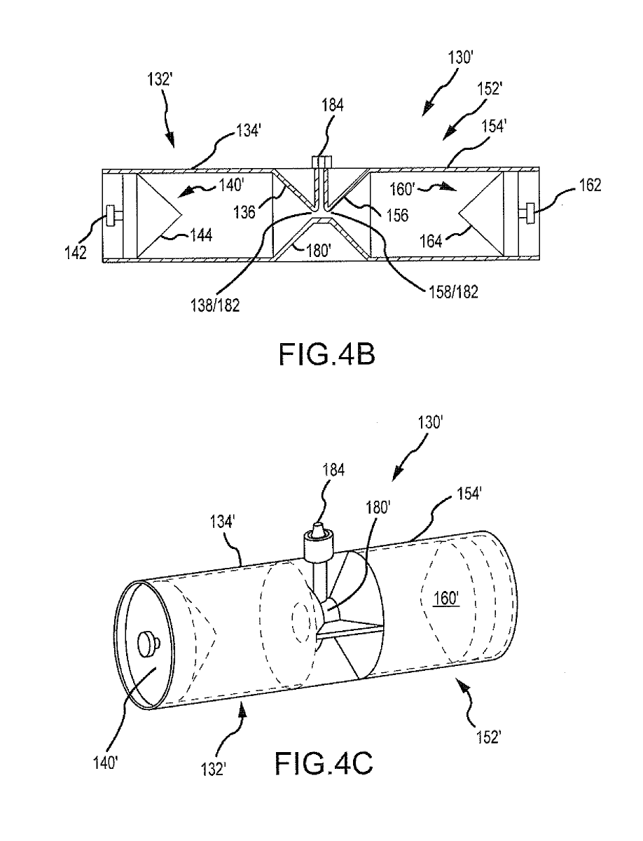

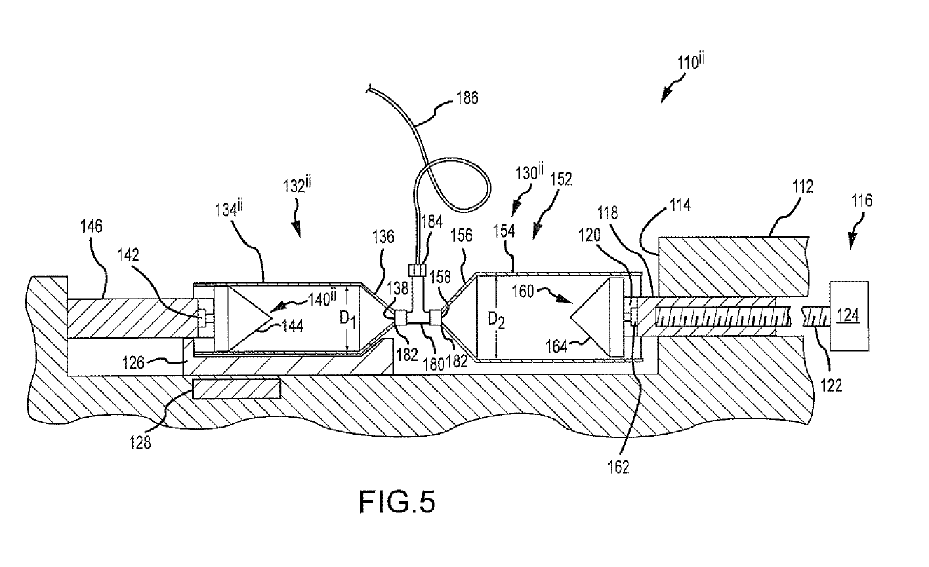

The first housing 134 of the first syringe 132, the second housing 154 of the second syringe 152, and the connector 180 may be an integrally-formed structure (e.g., of one-piece construction). In this case, there would be no joint of any kind between the connector 180 and either of the first housing 134 and the second housing 154. However, the first housing 134, second housing 154, and connector 180 could be separately formed structures that are appropriately interconnected (e.g., in accordance with the power injector 110ⁱ of FIG. 4A; the power injector 110ⁱⁱ of FIG. 5). In any case, the first housing 134, the connector 180, and the second housing 154 remain in an at least substantially fixed position relative to each other at all times in the case of the power injector 110, at least along the path of relative motion that provides a fluid discharge from either the first housing 134 or the second housing 154.

A default discharge sequence for the power injector 110 of FIGS. 3A-B is provided by a valve 170. This valve 170 is contained within the connector 180, and includes a valve head 172 and a biasing member 174. The valve head 172 may be of any appropriate size, shape, configuration, and/or type so as to be able to selectively block a flow out of the second outlet 158 for the second syringe 152. The biasing member 174 may be of any appropriate size, shape, configuration, and/or type so as to exert a certain force on the valve head 172 so as to block the flow out of the second outlet 158 until the development of a certain pressure within the second housing 154. Generally, the biasing number 174 itself exerts a sufficient force on the valve head 172 so as to block a flow out of the second syringe 152, regardless of the pressure within the first syringe 132. That is, a biasing force (provided by the biasing member 174), that is independent of the pressure within the first housing 134 of the first syringe 132, fluidly isolates the second housing 154 from the connector 180 until a certain pressure develops within the second housing 154. In the illustrated embodiment, the biasing member 174 is in the form of a spring. It should be appreciated that the biasing force provided by the biasing member 174 (e.g., the spring force) and the area of the valve head 172 that is exposed to the fluid pressure within the second housing 154 each contribute to and/or otherwise affect the pressure at which the valve head 172 will “unseat” to allow a fluid discharge from the second housing 154. It should further be appreciated that this biasing force and/or surface area may be selected to realize a desired pressure at which the valve head 172 will “unseat” to allow a fluid discharge from the second housing 154.

Certain applications may benefit from being able to change the default discharge sequence utilized by the power injector 110 via the valve 170. In this regard, the power injector 110 includes a brake 128 that at least operatively interacts with the first housing 134 of the first syringe 132. Generally, the brake 128 may be of any appropriate size, shape, configuration, and/or type so as to be able to selectively terminate the motion of the first housing 134 during operation of the drive train 116. In the illustrated embodiment, the brake 128 is of an electromagnetic type that magnetically interacts with the syringe carriage 126, in which the first syringe housing 134 is disposed and as noted above. However and in accordance with the foregoing, the brake 128 could mechanically interact with the first housing 134 and/or the syringe carriage 126 to provide a braking function.

How fluid is discharged by the power injector 110 will now be summarized with continued reference to FIGS. 3A-B. Operation of the drive train 116 entails at least one drive source 124 rotating the single drive screw 122. Rotation of the drive screw 122 causes the single ram 118 to move along the drive screw 122 (along an axial path in the illustrated embodiment). This motion will be transferred to the second plunger 160 through any appropriate interaction with the ram 118 (a mechanical coupling in the illustrated embodiment). Based upon the diameter D₂ of the second housing 152 being at least as large as the diameter D₁ of the first housing 132, and furthermore based upon the valve 170 being biased to a closed position until there is at least a certain pressure buildup within the second housing 152, the noted movement of the ram 118 will collectively move the second plunger 160, the second housing 154, the connector 180, the syringe carriage 126, and the first housing 134 (to the left in the view shown in FIG. 3A) relative to the stationary first plunger 140 (based upon an appropriate interaction between the first plunger 140 and the stationary ram 146—a mechanical coupling in the illustrated embodiment). That is, the second plunger 160, the second housing 154, the connector 180, the syringe carriage 126, and the first housing 134 remain in an at least substantially fixed position relative to each other at this time. As such, there is a relative movement between the stationary first plunger 140 and the first housing 134 such that fluid is discharged from the first housing 134 through its first outlet 138, into the connector 180, and into the conduit 186.

Once the first plunger 140 reaches the end of the first housing 134, or once the first plunger 140 “bottoms out” against the first housing 134 (e.g., at the completion of the discharge stroke for the first syringe 132), the above-noted collective movement of the second plunger 160, the second housing 154, the connector 180, the syringe carriage 126, and the first housing 134 terminates. Since the ram 118 may continue to move at this time, the second plunger 160 will now begin to move (to the left in the view presented in FIG. 3A) relative to the now stationary second housing 154. Since the valve 170 is biased to close the second outlet 158 of the second syringe 152, this relative movement between the second plunger 160 and the second housing 154 may not initially generate a sufficient pressure within the second housing 154 to unseat the valve 170. As such, fluid may not be discharged from the second housing 154 at this time. After the force created by the increasing fluid pressure within the second housing 154 and acting on the valve head 172 exceeds the sum of the biasing force being exerted on the valve head 172 by the biasing number 174, along with any force being exerted on the valve head 172 by the fluid pressure within the first housing 134, the valve 170 will open (the valve head 172 will move away from the second outlet 158, or to the left in the view shown in FIG. 3A). Opening the valve 170 initiates a discharge of fluid from the second housing 154.

The drive train 116 may continually operate in the same configuration to either move the first plunger 140 relative to the first housing 134, or to move the second plunger 160 relative to the second housing 154. No change of any type in the drive train 116 is required for the drive train 116 to move the first plunger 140 relative to the first housing 134, and for the same to move the second plunger 160 relative to the second housing 154. There will either be relative movement between the first plunger 140 and the first housing 134, or relative movement between the second plunger 160 and the second housing 154, at all times during an operation of the drive train 116 in a direction that produces a fluid discharge (a movement of the ram 118 to the left in the view shown in FIG. 3A) and when there is an appropriate interaction between the ram 118 and the second plunger 160. As discussed above and in accordance the default discharge sequence for the power injector 110, all fluid may be discharged from the first housing 134 (by a relative movement between the stationary first plunger 140 and an axially moving first housing 134), before any fluid is discharged from the second housing 154. The power injector 110 may be configured to at least temporarily/selectively operate other than in accordance with this default discharge sequence.

The power injector 110 includes the syringe carriage 126 that carries the first housing 134 and that is able to move relative to the powerhead 112 (e.g., along an axial path). The powerhead 112 may include a brake 128 that at least operatively interacts with the syringe carriage 126 (in which the first housing 134 of the first syringe 132 is disposed). Operation of this brake 128 may be used to change the default discharge sequence. One characterization of the brake 128 is that it may be changed from a disengaged configuration to an engaged configuration, as well as from an engaged configuration back to a disengaged configuration. The disengaged configuration of the brake 128 (e.g., a first brake configuration) allows the syringe carriage 126 (and thereby the first housing 134) to move relative to the powerhead 112, while the engaged configuration of the brake 128 (e.g., a second brake configuration) maintains the syringe carriage 126 (and thereby the first housing 134) in an at least substantially fixed position relative to the powerhead 112.

The brake 128 may be operated at any time prior to and/or during operation of the drive train 116 to modify the default discharge sequence of the power injector 110. Consider the case where the drive train 116 is being operated in a first configuration to move the stationary first plunger 140 relative to the first housing 134 to provide a fluid discharge from the first housing 134, where the first plunger 140 has not yet completed its discharge stroke (e.g., the first plunger 140 has not yet “bottomed out” on the end of the first housing 134), and where no fluid has yet been discharged from the second housing 154. The brake 128 may be changed from its disengaged configuration to its engaged configuration to terminate the motion of the first housing 134, such that the first plunger 140 and the first housing 134 will then remain in an at least substantially fixed position relative to each other. This terminates the fluid discharge from the first housing 134. Since the first housing 134 and the second housing 154 are maintained in an at least substantially fixed position relative to each other, the termination of the movement of the first housing 134 by the brake 128 also terminates the movement of the second housing 154. Continued/further operation of the drive train 116 will then move the second plunger 160 relative to the now stationary second housing 154 to discharge fluid from the second housing 154. This change in fluid discharge from the first housing 134 to the second housing 154 requires no change of any type in the configuration of the drive train 116, nor does it require any suspension of its operation. The ram 118 simply continues to move along the rotating drive screw 122. Although suspension of the operation of the drive train 116 is not required to change the discharge from one of the first housing 134 and the second housing 154 to the other, the operation of the drive train 116 may of course be suspended for any appropriate reason.

It should be appreciated that the brake 128 could be operated in accordance with the foregoing at the time of or prior to initiating operation of the drive train 116, such that the initial discharge from the power injector 110 is provided from the second housing 154. It should also be appreciated that the state or the configuration of brake 128 may be changed any number of times to change the discharge from the first housing 134 to the second housing 154, and vice versa. When the brake 128 is in its disengaged configuration or state, the power injector 110 will revert to its default discharge sequence, where remaining fluid will be discharged from the first housing 134 prior to then initiating a subsequent fluid discharge from the second housing 154.

Another embodiment of a power injector is identified by reference numeral 110ⁱ, and is illustrated in FIG. 4A. Corresponding components between the power injector 110 of FIG. 3A and the power injector 110ⁱ of FIG. 4A are identified by the same reference numerals. Those corresponding components that differ in at least some respect are further identified by a superscripted “i” in relation to the power injector 110ⁱ of FIG. 4A. The primary difference between the embodiment of FIGS. 3A and 4A is that the power injector 110ⁱ of FIG. 4A does not include the valve 170 to provide a default discharge sequence, as does the power injector 110 of FIG. 3A. Instead, changes have been made to the syringe assembly 130ⁱ, specifically in relation to the first syringe 132ⁱ and the second syringe 152.

The power injector 110ⁱ uses a different frictional interaction between the first plunger 140ⁱ/first housing 134ⁱ and the second plunger 160ⁱ/second housing 154ⁱ to provide a default discharge sequence (versus the valve 170 in the case of the power injector 110 of FIG. 3A). More specifically, there is a first frictional interaction or a first frictional force between the first plunger 140ⁱ and the first housing 134ⁱ along the axis of relative movement between these components (double-headed arrow A), while there is a second frictional interaction or a second frictional force between the second plunger 160ⁱ and the second housing 154ⁱ along the path of relative movement between these components (double-headed arrow B). In the illustrated embodiment of FIG. 4A, the magnitude of the first frictional interaction or first frictional force between the first plunger 140ⁱ and the first housing 134ⁱ is less than the magnitude of the second frictional interaction or second frictional force between the second plunger 160ⁱ and the second housing 154ⁱ. The differences of the frictional interactions/forces between the first plunger 140ⁱ/first housing 134ⁱ and the second plunger 160ⁱ/second housing 154ⁱ may be realized in any appropriate manner,

In the case of the power injector 110ⁱ of FIG. 3A, the first housing 134ⁱ and the second housing 154ⁱ are of the same size. This may be characterized as the inner diameter D₁ of the first housing 134ⁱ and the inner diameter D₂ of the second housing 154ⁱ being of the same magnitude. This may also be characterized as the outer diameter of the first plunger 140ⁱ being the same as the outer diameter of the second plunger 160ⁱ.

How fluid is discharged by the power injector 110ⁱ will now be summarized with continued reference to FIG. 4A. Operation of the drive train 116 entails at least one drive source 124 rotating the single drive screw 122. Rotation of the drive screw 122 causes the single ram 118 to move along the drive screw 122 (along an axial path in the illustrated embodiment). This motion will be transferred to the second plunger 160 through any appropriate interaction with the ram 118 (a mechanical coupling in the illustrated embodiment). Based upon the first frictional interaction or first frictional force between the first plunger 140ⁱ and the first housing 134ⁱ being less than the second frictional interaction or second frictional force between the second plunger 160ⁱ and the second housing 154ⁱ, the noted movement of the ram 118 will collectively move the second plunger 160ⁱ, the second housing 154ⁱ, the connector 180, the syringe carriage 126, and the first housing 134ⁱ (to the left in the view shown in FIG. 4A) relative to the stationary first plunger 146 (based upon an appropriate interaction between the first plunger 140 and the stationary ram 146—a mechanical coupling in the illustrated embodiment). That is, the second plunger 160ⁱ, the second housing 154ⁱ, the connector 180, the syringe carriage 126, and the first housing 134ⁱ remain in an at least substantially fixed position relative to each other at this time. As such, there is a relative movement between the stationary first plunger 140ⁱ and the first housing 134ⁱ such that fluid is discharged from the first housing 134ⁱ through its first outlet 138, into the connector 180, and into the conduit 186.

Once the first plunger 140ⁱ reaches the end of the first housing 134ⁱ, or once the first plunger 140ⁱ “bottoms out” against the first housing 134ⁱ (e.g., at the completion of the discharge stroke for the first syringe 132ⁱ), the above-noted collective movement of the second plunger 160ⁱ, the second housing 154ⁱ, the connector 180, the syringe carriage 126, and the first housing 134ⁱ terminates. Since the ram 118 may continue to move at this time, the second plunger 160ⁱ will now begin to move (to the left in the view presented in FIG. 4A) relative to the now stationary second housing 154ⁱ to provide a fluid discharge from the second housing 154ⁱ.

The drive train 116 may continually operate in the same configuration to either move the first plunger 140ⁱ relative to the first housing 134ⁱ, or to move the second plunger 160ⁱ relative to the second housing 154ⁱ in the case of the power injector 110ⁱ of FIG. 4A. No change of any type in the drive train 116 of the power injector 110ⁱ is required for the drive train 116 to move the first plunger 140ⁱ relative to the first housing 134ⁱ, and for the same to move the second plunger 160ⁱ relative to the second housing 154ⁱ. There will either be relative movement between the first plunger 140ⁱ and the first housing 134ⁱ, or relative movement between the second plunger 160ⁱ and the second housing 154ⁱ, at all times during an operation of the drive train 116 in a direction that produces a fluid discharge (a movement to the ram 118 to the left in the view shown in FIG. 4A) and when there is an appropriate interaction between the ram 118 and the second plunger 160ⁱ.

As discussed above, the default discharge sequence for the power injector 110ⁱ of FIG. 4A is for all fluid to be discharged from the first housing 134ⁱ (by a relative movement between the stationary first plunger 140ⁱ and an axially moving first housing 134ⁱ), before any fluid is discharged from the second housing 154ⁱ. Again, the default discharge sequence is provided through different frictional interactions between the first plunger 140ⁱ/first housing 134ⁱ and between the second plunger 160ⁱ/second housing 154ⁱ in the case of the power injector 110ⁱ. The power injector 110ⁱ is also configurable to operate other than in accordance with this default discharge sequence. As in the same manner as the power injector 110 of FIG. 3A, the brake 128 of the power injector 110ⁱ may be operated to change its friction-based default discharge sequence. With the brake 128 in an engaged state or condition, fluid will be discharged from the second housing 154ⁱ. With the brake 128 in a disengaged state or condition, and assuming the first plunger 140ⁱ has not “bottomed out” at the end of the first housing 134ⁱ, fluid will be discharged from the first housing 134ⁱ.

The syringe assembly 130 used by the power injector 110 of FIG. 3A and the syringe assembly 130ⁱ used by the power injector 110ⁱ of FIG. 4A each use a separate first housing 1341134ⁱ, a separate second housing 154/154ⁱ, and a separate connector 180. That is, there is a joint between the first housing 134/134ⁱ and the connector 180, and there is a joint between the second housing 154/154ⁱ and the connector 180. This feature may be utilized to change out one or more of these three components for any appropriate reason. Another option is for these three components to be part of a common structure. One embodiment of a syringe assembly 130ⁱ that may be utilized by each of the power injectors 110, 110ⁱ is illustrated in FIGS. 4B and 4C. In the case of the syringe assembly 130ⁱ, the first housing 134ⁱ, the second housing 154ⁱ, and the connector 180ⁱ are part of a single, integrally formed structure. That is, there is no joint of any kind between the first housing 134ⁱ and the connector 180ⁱ, nor is there a joint of any kind between the second housing 154ⁱ and the connector 180ⁱ. However, the syringe assembly 130ⁱ may still be characterized as including a first syringe 132ⁱ and a second syringe 152ⁱ. Moreover, a first plunger 140ⁱ is still movably disposed within the first housing 134ⁱ, and a second plunger 160ⁱ is still movably disposed within the second housing 154ⁱ.

Another embodiment of a power injector is identified by reference numeral 110ⁱⁱ, and is illustrated in FIG. 5. Corresponding components between the power injector 110 of FIG. 3A and the power injector 110ⁱⁱ of FIG. 5 are identified by the same reference numerals. Those corresponding components that differ in at least some respect are further identified by a superscripted “ii” in relation to the power injector 110ⁱⁱ of FIG. 5. The primary difference between the embodiment of FIGS. 3A and 5 is that the power injector 110ⁱⁱ of FIG. 5 does not include the valve 170 to provide the default discharge sequence, as does the power injector 110 of FIG. 3A. Instead, changes have been made to the syringe assembly 130ⁱⁱ, specifically in relation to the first syringe 132ⁱ.

The power injector 110ⁱⁱ, incorporates a selective sizing of the first housing 134ⁱⁱ and the second housing 154 to provide a default discharge sequence (versus the valve 170 in the case of the power injector 110 of FIG. 3A). The first housing 134ⁱⁱ, is smaller than the second housing 154 in the case of the power injector 110ⁱⁱ of FIG. 5. This may be characterized as the inner diameter D₁ of the first housing 134ⁱⁱ being smaller than the inner diameter D₂ of the second housing 154. This may also be characterized as the outer diameter of the first plunger 140ⁱⁱ, being smaller than the outer diameter of the second plunger 160. Since the first housing 134ⁱⁱ, is smaller than the second housing 154, the default discharge sequence for the power injector 110ⁱⁱ is that fluid is discharged from the first housing 134ⁱⁱ prior to fluid being discharged from the second housing 154. The syringe assembly 130ⁱ illustrated in FIGS. 48 and 4C could be adapted for use by the power injector 110ⁱⁱ (incorporating the described differential sizing between the first housing 134ⁱⁱ and the second housing 154).

How fluid is discharged by the power injector 110ⁱ will now be summarized with continued reference to FIG. 5. Initially, it should be noted that the pressure in the first housing 134ⁱⁱ will be the same as the pressure within the second housing 154 prior to operating the drive train 116 there is open fluid communication between the connector 180 and each of the first housing 134ⁱⁱ and the second housing 154. Operation of the drive train 116 entails at least one drive source 124 rotating the single drive screw 122. Rotation of the drive screw 122 causes the single ram 118 to move along the drive screw 122 (along an axial path in the illustrated embodiment). This motion will be transferred to the second plunger 160 through any appropriate interaction with the ram 118 (a mechanical coupling in the illustrated embodiment). Since the first housing 134ⁱⁱ is smaller than the second housing 154 (the first plunger 140ⁱⁱ being smaller than the second plunger 160), it takes less force to move the first plunger 140ⁱⁱ relative to the first housing 134ⁱⁱ than to move the second plunger 160 relative to the second housing 154. As such, the noted movement of the ram 118 will collectively move the second plunger 160, the second housing 154, the connector 180, the syringe carriage 126, and the first housing 134ⁱⁱ (to the left in the view shown in FIG. 5) relative to the stationary first plunger 140ⁱⁱ (based upon an appropriate interaction between the first plunger 140ⁱⁱ and the stationary ram 146—a mechanical coupling in the illustrated embodiment). That is, the second plunger 160, the second housing 154, the connector 180, the syringe carriage 126, and the first housing 134ⁱⁱ remain in an at least substantially fixed position relative to each other at this time. As such, there is a relative movement between the stationary first plunger 140ⁱⁱ and the first housing 134ⁱⁱ such that fluid is discharged from the first housing 134ⁱⁱ through its first outlet 138, into the connector 180, and into the conduit 186.

Once the first plunger 140ⁱⁱ reaches the end of the first housing 134ⁱⁱ, or once the first plunger 140ⁱⁱ “bottoms out” against the first housing 134ⁱⁱ (e.g., at the completion of the discharge stroke for the first syringe 132ⁱⁱ), the above-noted collective movement of the second plunger 160, the second housing 154, the connector 180, the syringe carriage 126, and the first housing 134ⁱⁱ terminates. Since the ram 118 may continue to move at this time, the second plunger 160ⁱⁱ will now begin to move (to the left in the view presented in FIG. 5) relative to the now stationary second housing 154 to provide a fluid discharge from the second housing 154.

The drive train 116 may continually operate in the same configuration to either move the first plunger 140ⁱⁱ relative to the first housing 134ⁱⁱ, or to move the second plunger 160 relative to the second housing 154 in the case of the power injector 110ⁱ of FIG. 5. No change of any type in the drive train 116 of the power injector 110ⁱⁱ is required for the drive train 116 to move the first plunger 140ⁱⁱ relative to the first housing 134ⁱⁱ, and for the same to move the second plunger 160 relative to the second housing 154. There will either be relative movement between the first plunger 140ⁱⁱ and the first housing 134ⁱⁱ, or relative movement between the second plunger 160 and the second housing 154, at all times during an operation of the drive train 116 in a direction that produces a fluid discharge (a movement of the ram 118 to the left in the view shown in FIG. 5) and when there is an appropriate interaction between the ram 118 and the second plunger 160.

As discussed above, the default discharge sequence for the power injector 110ⁱⁱ of FIG. 5 is for all fluid to be discharged from the first housing 134ⁱⁱ (by a relative movement between the stationary first plunger 140ⁱⁱ and an axially moving first housing 134ⁱⁱ), before any fluid is discharged from the second housing 154. Again, the default discharge sequence is provided through the first plunger 140ⁱⁱ/first housing 134ⁱⁱ being smaller than the corresponding one of the second plunger 160 and the second housing 154 in the case of the power injector 110ⁱⁱ. The power injector 110ⁱⁱ is also configurable to operate other than in accordance with this default discharge sequence. As in the same manner as the power injector 110 of FIG. 3A, the brake 128 of the power injector 110ⁱⁱ may be operated to change its sizing-based default discharge sequence. With the brake 128 in an engaged condition or state, fluid will be discharged from the second housing 154. With the brake 128 in a disengaged state or condition, and assuming the first plunger 140ⁱⁱ has not “bottomed out” at the end of the first housing 134ⁱⁱ, fluid will be discharged from the first housing 134ⁱⁱ.

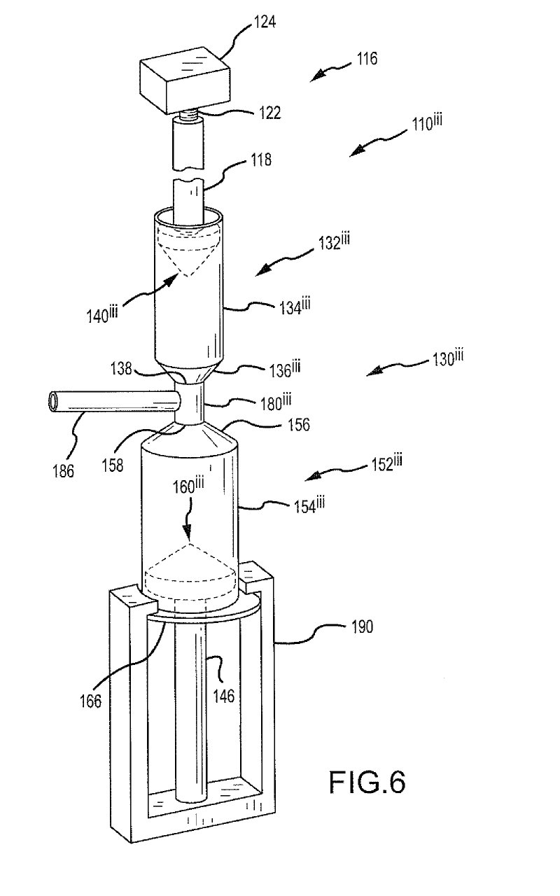

Another embodiment of a power injector is identified by reference numeral 110ⁱⁱ, and is illustrated in FIG. 6. Corresponding components between the power injector 110 of FIG. 3A and the power injector 110ⁱⁱ of FIG. 6 are identified by the same reference numerals. Those corresponding components that differ in at least some respect are further identified by a superscripted “iii” in relation to the power injector 110ⁱⁱⁱ of FIG. 6. The illustrated syringe assembly 130ⁱⁱⁱ for the power injector 110ⁱⁱⁱ is an integral structure as described above in relation to FIGS. 4B and 4C. That is, there is no joint of any kind between the first housing 134ⁱⁱⁱ and the connector 180ⁱⁱⁱ, nor is there a joint of any kind between the second housing 154ⁱⁱⁱ and the connector 180ⁱⁱⁱ. However, the first housing 134ⁱⁱⁱ, the connector 180ⁱⁱⁱ, and the second housing 154ⁱⁱⁱ could each be separately formed structures that are appropriately attached/interconnected to define the syringe assembly 130ⁱⁱⁱ (not shown).

The power injector 110ⁱⁱⁱ of FIG. 6 may at first glance appear to be similar to the power injector 110ⁱⁱⁱ of FIG. 5. For instance, the first housing 134ⁱⁱⁱ of the first syringe 132ⁱⁱⁱ is smaller (e.g., has a smaller inner diameter) than the second housing 154ⁱⁱⁱ of the second syringe 152ⁱⁱⁱ, or stated another way the outer diameter of the first plunger 140ⁱⁱⁱ is smaller than that of the second plunger 160ⁱⁱⁱ. However and in direct contrast to the power injector 110ⁱⁱⁱ of FIG. 5, the first plunger 140ⁱⁱⁱ is interconnected with a movable, single ram 118, while the second plunger 160ⁱⁱⁱ is interconnected with a stationary ram 146 in the case of the power injector 110ⁱⁱⁱ. The power injector 110ⁱⁱⁱ also does not include a brake 128. Instead, the power injector 110ⁱⁱⁱ includes a back-drive resistor 190.

The back-drive resistor 190 at least operatively interacts with the second housing 154ⁱⁱⁱ in the case of the power injector 110ⁱⁱⁱ of FIG. 6. In the illustrated embodiment, there is actually a mechanical interaction between the back-drive resistor 190 and the second housing 154ⁱⁱⁱ. Generally, the back-drive resistor 190 may be viewed as limiting the amount that the second plunger 160ⁱⁱⁱ may be retracted relative to the second housing 154ⁱⁱⁱ. The “fully retracted” position for the second plunger 160ⁱⁱⁱ may be characterized as being associated with the maximum fluid volume that may be discharged for the case of the second housing 154ⁱⁱⁱ, or where the spacing between the second plunger 160ⁱⁱⁱ and the second outlet 158 is at a maximum (the FIG. 6 position). The back-drive resistor 190 may define such a fully retracted position. In contrast, the fully extended position for the second plunger 160ⁱⁱⁱ may be characterized as being where the second plunger 160ⁱⁱⁱ is adjacent to the second outlet 158, or where the spacing between the second plunger 160ⁱⁱⁱ and the second outlet 158 is at a minimum.

When the second plunger 160ⁱⁱⁱ is in its fully retracted position and during operation of the drive train 116, the back-drive resistor 190 may provide a resistive force that opposes an attempted movement of the first housing 134ⁱⁱⁱ and the second housing 154ⁱⁱⁱ in a direction that is directly opposite of the direction that the first plunger 140ⁱⁱⁱ is moving during operation of the drive train 116 to discharge fluid from the first housing 134ⁱⁱⁱ. That is, during operation of the drive train 116 where the first plunger 140ⁱⁱⁱ is moving in the downward direction in the view shown in FIG. 6, the first housing 134ⁱⁱⁱ and the second housing 154ⁱⁱⁱ may attempt to move in an upward direction in the view shown in FIG. 6. Any such movement by the first housing 134ⁱⁱⁱ and second housing 154ⁱⁱⁱ would be undesirable in a number of respects. First, this movement would enhance the degree of relative movement between the first plunger 140ⁱⁱⁱ and the first housing 134ⁱⁱⁱ in a manner that would increase the discharge rate from the first housing 134ⁱⁱⁱ. Second, this movement would attempt to “blow” the second plunger 160ⁱⁱⁱ out of the back end of the second housing 154ⁱⁱⁱ. However, this movement of the first housing 134ⁱⁱⁱ and the second housing 154ⁱⁱⁱ is opposed and resisted by the back-drive resistor 190. The opposition/resistive force provided by the back-drive resistor 190 preferably maintains the first housing 134ⁱⁱⁱ and the second housing 154ⁱⁱⁱ in a fixed position until the first plunger 140ⁱⁱⁱ “bottoms out” on the end of the first housing 134ⁱⁱⁱ, or until the first plunger 140ⁱⁱⁱ completes its discharge stroke.

The back-drive resistor 190 may be a structure or combination of structures of any appropriate size, shape, configuration, and/or type that provides the above-noted opposition/resistive force to movement of the first housing 134ⁱⁱⁱ and the second housing 154ⁱⁱⁱ. In the illustrated embodiment, the back-drive resistor 190 is in the form an external stop, abutment, or the like that is engageable with an external flange 166 of the second housing 154ⁱⁱⁱ. The position of the back-drive resistor 190 could be adjustable along the axis of relative motion between the first plunger 140ⁱⁱⁱ and the first housing 134ⁱⁱⁱ, which also coincides with the axis of relative motion between the second plunger 160ⁱⁱⁱ and the second housing 154ⁱⁱⁱ. The back-drive resistor 190 could also be in the form of a stop, abutment, or the like on an interior surface of the second housing 154ⁱⁱⁱ to limit the range of relative motion between the second plunger 160ⁱⁱⁱ and the second housing 154ⁱⁱⁱ by being engageable with the second plunger 160ⁱⁱⁱ, for instance the “back side” of the head of the second plunger 160ⁱⁱⁱ. A sufficiently large frictional interface between the second housing 154ⁱⁱⁱ and the second plunger 160ⁱⁱⁱ may define the back-drive resistor 190. Another option would be for the back-drive resistor 190 to be in the form of a one-way ratchet between the second plunger 160ⁱⁱⁱ and the second housing 154ⁱⁱⁱ.

How fluid is discharged by the power injector 110ⁱⁱⁱ will now be summarized with continued reference to FIG. 6. The pressure in the first housing 134ⁱⁱⁱ should be the same as the pressure within the second housing 154ⁱⁱⁱ prior to operating the drive train 116ⁱⁱⁱ there is open fluid communication between the connector 180ⁱⁱⁱ and each of the first housing 134ⁱⁱⁱ and the second housing 154ⁱⁱⁱ. Operation of the drive train 116 entails at least one drive source 124 rotating the single drive screw 122. Rotation of the drive screw 122 causes the single ram 118 to move along the drive screw 122 (along an axial path in the illustrated embodiment). This motion will be transferred to the first plunger 140ⁱⁱⁱ through any appropriate interaction with the ram 118 (a mechanical coupling in the illustrated embodiment). Since the first housing 134ⁱⁱⁱ is smaller than the second housing 154ⁱⁱⁱ (the first plunger 140ⁱⁱⁱ being smaller than the second plunger 160ⁱⁱⁱ), it takes less force to move the first plunger 140ⁱⁱⁱ relative to the first housing 134ⁱⁱⁱ than to move the second plunger 160ⁱⁱⁱ relative to the second housing 150. As such, the noted movement of the ram 118 will move the first plunger 140ⁱⁱⁱ relative to a stationary first housing 134ⁱⁱⁱ such that fluid is discharged from the first housing 134ⁱⁱⁱ through its first outlet 138, into the connector 180ⁱⁱⁱ, and into the conduit 186. As the position of the first housing 134ⁱⁱⁱ remains fixed relative to each of the second housing 154ⁱⁱⁱ and the connector 180ⁱⁱⁱ, the second housing 150 and the connector 180ⁱⁱⁱ will also remain stationary at this time.

During the above-noted movement of the first plunger 140ⁱⁱⁱ toward the first outlet 138 of the first housing 134ⁱⁱⁱ, a biasing force may be exerted on the combined assembly of the first housing 134ⁱⁱⁱ, the connector 180ⁱⁱⁱ, and the second housing 150. The vector of this biasing force would be in the direction opposite of the direction that the first plunger 140ⁱⁱⁱ is moving at this time. In the view presented in FIG. 6, any such biasing force would attempt to move the first housing 134ⁱⁱⁱ, the connector 180ⁱⁱⁱ, and the second housing 154ⁱⁱⁱ in an upward direction (while the first plunger 140ⁱⁱⁱ is moving in a downward direction in this same view). The back-drive resistor 190 would exert a force on the second housing 150 in this instance to counter or directly oppose this biasing force to maintain the first housing 134ⁱⁱⁱ, the connector 180ⁱⁱⁱ, and the second housing 150 in a stationary position. That is, the vector of the force being exerted on the second housing 154ⁱⁱⁱ by the back-drive resistor 190 would be in the “downward” direction in the view shown in FIG. 6.

Once the first plunger 140ⁱⁱⁱ reaches the end of the first housing 134ⁱⁱⁱ, or once the first plunger 140ⁱⁱⁱ “bottoms out” against the first housing 130 (e.g., at the completion of the discharge stroke for the first syringe) 132ⁱⁱⁱ), the above-noted movement of the first plunger 140ⁱⁱⁱ relative to the first housing 134ⁱⁱⁱ will terminate. Continued operation of the drive train 116 (without having to change its configuration in any manner) will then collectively move the first plunger 140ⁱⁱⁱ, the first housing 134ⁱⁱⁱ, the second housing 154ⁱⁱⁱ, and the connector 180ⁱⁱⁱ relative to the stationary second plunger 160ⁱⁱⁱ to now discharge fluid from the second housing 154ⁱⁱⁱ (in a downward direction in the view shown in FIG. 6).

The drive train 116 may continually operate in the same configuration to either move the first plunger 140ⁱⁱⁱ relative to a then stationary first housing 134ⁱⁱⁱ, or to move the then stationary second plunger 160ⁱⁱⁱ relative to the moving second housing 154ⁱⁱⁱ in the case of the power injector 110ⁱⁱⁱ of FIG. 6. No change of any type in the drive train 116 of the power injector 110ⁱⁱⁱ is required for the drive train 116 to move the first plunger 140ⁱⁱⁱ relative to the first housing 134ⁱⁱⁱ, and for the same to move the second plunger 160ⁱⁱⁱ relative to the second housing 154ⁱⁱⁱ. There will either be relative movement between the first plunger 140ⁱⁱⁱ and the first housing 134ⁱⁱⁱ, or relative movement between the second plunger 160ⁱⁱⁱ and the second housing 154ⁱⁱⁱ, at all times during an operation of the drive train 116 in a direction to produce fluid discharges (a movement of the ram 118 in a downward direction in the view shown in FIG. 6) and when there is an appropriate interaction between the ram 118 and the first plunger 140ⁱⁱⁱ.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

1-95. (canceled)

96. A power injector, comprising:
a powerhead that comprises:
a drive train having a movable ram designed to advance axially in a first direction and a second direction opposite of the first direction; and
a stationary ram; and
a syringe assembly mounted to the powerhead and comprising:
a first housing that is movable relative to said stationary ram;
a first plunger disposed within and movable relative to said first housing, wherein said first plunger is in contact with said stationary ram of said powerhead;
a second housing that is movable relative to said stationary ram of said powerhead; and
a second plunger disposed within and movable relative to said second housing, wherein the second plunger is in contact with said movable ram of said drive train,
wherein one part of an operation of said drive train in a first configuration moves said first plunger relative to said first housing, wherein another part of said operation of said drive train in said first configuration moves said second plunger relative to said second housing.

97. The power injector of claim 96, further comprising:
a discharge sequence controller, wherein said discharge sequence controller allows a discharge from said syringe assembly to be changed from said first housing to said second housing and from second housing to said first housing.

98. The power injector of claim 96, further comprising:
a brake, wherein changing a state of said brake changes a discharge sequence for said power injector between said first and second housings, and wherein said state of said brake is changed by a non-mechanical signal.

99. The power injector of claim 96, further comprising:
a brake operatively interconnected with said first housing of said syringe assembly, wherein engagement of said brake maintains said first housing in a fixed position, and wherein said brake may be changed from an engaged configuration to an disengaged configuration and from said disengaged configuration to said engaged configuration.

100. The power injector of claim 96, further comprising:
a brake disposable in first and second brake configurations, wherein said first brake configuration allows said first housing and said first plunger to move collectively during operation of said drive train, and wherein said second brake configuration allows said first plunger to move relative to said first housing during said operation of said drive train.

101. The power injector of claim 96, further comprising:
a valve; and
a first force that biases said valve to a position where said valve blocks a flow out of said second housing, wherein said first force is separate from and independent of a pressure in said first housing.

102. The power injector of claim 101, wherein said valve is movable between first and second positions.

103. The power injector of claim 101, wherein said valve is movable in response to a differential pressure between said first and second housings.

104. The power injector of claim 101, wherein said valve determines a default order in which fluid is discharged from said first and second housings by said operation of said drive train in said first configuration.

105. The power injector of claim 96, wherein a first frictional interaction exists between said first housing and said first plunger, a second frictional interaction exists between said second housing and said second plunger, and a magnitude of said first frictional interaction is different than a magnitude of said second frictional interaction.

106. The power injector of claim 105, wherein said first frictional interaction is less than said second frictional interaction such that a default discharge sequence is from said first housing, followed by from said second housing.

107. The power injector of claim 105, wherein a sequence of discharges from said first and second housings is dependent upon said magnitudes of said first and second frictional interactions.

108. The power injector of claim 105, wherein said one part of said operation of said drive train in said first configuration precedes said another part of said operation of said drive train in said first configuration based upon said magnitude of said first frictional interaction being less than said magnitude of said second frictional interaction.

109. The power injector of claim 96, wherein said first and second housings have a common inner diameter.

110. The power injector of claim 96, wherein the first housing comprises a first inner diameter, the second housing is collectively movable with said first housing and comprises a second inner diameter larger than said first inner diameter, and said first and second housings are maintained in a fixed position relative to each other at all times.

111. The power injector of claim 96, further comprising:
a back-drive resistor operatively associated with said second housing.

112. The power injector of claim 111, wherein said back-drive resistor maintains said second plunger and said second housing in a fixed relative positional relationship during said one part of said operation of said drive train in said first configuration.

113. The power injector of claim 111, wherein said back-drive resistor provides a resistive force that opposes movement of said first and second housings in a direction that is directly opposite to a direction that said first plunger is moving during said one part of said operation of said drive train in said first configuration.

114. The power injector of claim 96, wherein said syringe assembly comprises:
a first syringe comprising said first housing, said first plunger, and a first outlet;
a second syringe comprising said second housing, said second plunger, and a second outlet; and
a separate connector fluidly interconnected with each of said first and second outlets.

115. The power injector of claim 96, further comprising:
an adjoining connector between and fluidly interconnected with each of said first and second housings, wherein said first housing comprises a first outlet, wherein said second housing comprises a second outlet, wherein said first and second outlets each fluidly communicate directly with said connector, and wherein said first housing, said connector, and said second housing are integrally formed.

116. The power injector of claim 96, wherein said first and second housings of said syringe assembly are maintained in a fixed position relative to each other at all times.

117. The power injector of claim 96, wherein said first and second housings of said syringe assembly are disposed in opposing relation.

118. The power injector of claim 96, wherein said first and second plungers are disposed along a common axis.

119. The power injector of claim 96, wherein a first relative movement between said first plunger and said first housing provides a discharge from said first housing, wherein a second relative movement between said second plunger and said second housing provides a discharge from said second housing, and wherein a spacing between said first and second plungers is reduced by each of said first and second relative movements.

120. The power injector of claim 96, wherein said first plunger is maintained in a fixed position at all times relative to the stationary ram.
